(19) United States
(12) Patent Application Publication
(10) Pub. No.: US 2009/0311182 A1
(43) Pub. Date: Dec. 17, 2009

(54) Macromolecular Delivery Systems for Non-Invasive Imaging, Evaluation and Treatment of Arthritis and Other Inflammatory Diseases

(76) Inventors: Dong Wang, Omaha, NE (US); Xinming Liu, Omaha, NE (US); Ling-Dong Quan, Omaha, NE (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/351,417

(22) Filed: Jan. 9, 2009

Related U.S. Application Data
(63) Continuation-in-part of application No. 10/591,258, filed on Nov. 28, 2006.
(60) Provisional application No. 60/558,047, filed on Mar. 31, 2004.
(60) Provisional application No. 61/010,595, filed on Jan. 10, 2008.
(60) Provisional application No. 61/134,310, filed on Jul. 9, 2008.

(51) Int. Cl. A61K 31/573; A61P 29/00; A61K 47/48; C07C 243/10; C07K 2/00; C08F 20/58; A61K 49/12

(52) U.S. Cl. 424/9.1; 514/772.4; 514/180; 564/148; 530/300; 526/304

(57) ABSTRACT

This invention relates to biotechnology, more particularly, to water-soluble polymeric delivery systems for the imaging, evaluation and/or treatment of rheumatoid arthritis and other inflammatory diseases. Using modern MR imaging techniques, the specific accumulation of macromolecules in arthritic joints in adjuvant-induced arthritis in rats is demonstrated. The strong correlation between the uptake and retention of the MR contrast agent labeled polymer with histopathological features of inflammation and local tissue damage demonstrates the practical applications of the macromolecular delivery system of the invention.

This application is a continuation-in-part of U.S. patent application Ser. No. 10/591,258, filed on Nov. 28, 2006, which is a §371 application of PCT/US2005/010801, filed Mar. 30, 2005, which in turn claims the benefit of U.S. Provisional Application No. 60/558,047, filed on Mar. 31, 2004. This application also claims priority under 35 U.S.C. §119(e) to U.S. Provisional Patent Application No. 61/010,595, filed on Jan. 10, 2008, and U.S. Provisional Patent Application No. 61/134,310, filed on Jul. 9, 2008. The foregoing applications are incorporated by reference herein.

This invention was made with government support under Grant No. R01AR053325 awarded by the National Institutes of Health. The government has certain rights in the invention.

### FIELD OF THE INVENTION

This invention relates to biotechnology, more particularly, to water-soluble polymeric delivery systems for non-invasive imaging, evaluation and treatment of arthritis and other inflammatory diseases.

### BACKGROUND OF THE INVENTION

Several publications and patent documents are cited throughout the specification in order to describe the state of the art to which this invention pertains. Each of these citations is incorporated herein by reference as though set forth in full.

Rheumatoid arthritis (RA) is the most common inflammatory arthritis, affecting about 1 percent of the general population worldwide. In United States, about 4.5% of people over the age of 55 people have been affected (Firestein, G. S., Etiology and Pathogenesis of Rheumatoid Arthritis. In Ruddy et al. (ed.) Kelley's Textbook of Rheumatology, 6^{th} Ed. W.B. Saunders Company, St. Louis, 1997, pp 921; McDuffie, F. C., Morbidity impact of rheumatoid arthritis in society, Am. J. Med. (1985) 78:1-5).

As a symmetric disease, RA usually involves the same joints on both sides of the body. Angiogenesis and microvascular lesions are common features of RA inflammation, which leads to abnormal serum protein infiltration into the synovia (Smolen and Steiner, Therapeutic strategies for rheumatoid arthritis, Nature Review Drug Discovery (2003) 2:473-488; Wallis et al., Protein traffic in human synovial effusion, Arthritis and Rheumatism (1987) 30:57-63; Levick, J. R., Permeability of rheumatoid and normal human synovium to specific plasma proteins, Arthritis and Rheumatism (1981) 24:1550-1560). Damaged or depleted lymphatics have been observed in the synovium of RA patients as well (Albuquerque and de Lima, Articular lymphoscintigraphy in human knees using radiolabeled dextran, Lymphology (1990) 23:215-218; Wilkinson and Edwards, Demonstration of lymphatics in human synovial tissue, Rheumatol. Int. (1991) 11:151-155).

Although the exact cause of rheumatoid arthritis is unknown, many medications have been developed to relieve its symptoms and slow or halt its progression. Most commonly used medications rest on three principal approaches: symptomatic treatment with non-steroidal anti-inflammatory drugs (NSAIDs), corticosteroid and disease-modifying antirheumatic drugs (DMARDs) (Smolen and Steiner, Therapeutic strategies for rheumatoid arthritis, Nature Review Drug Discovery (2003) 2:473-488).

Considerable effort has been made to identify and develop new therapeutic strategies for the treatment of RA. RA medications, such as cycloxygenase-2 (COX-2) specific inhibitor (a NSAID) (Matteson, E. L., Current treatment strategies for rheumatoid arthritis, Mayo Clin. Proc. (2000) 75:69-74), tumor necrosis factor (TNF) blockers and interleukin-1 receptor antagonists (IL-1Ra) (DMARDs) have been used for clinical applications (Smolen and Steiner, Therapeutic strategies for rheumatoid arthritis, Nature Review Drug Discovery (2003) 2:473-488) (Kitamura et al., AG-041R, a novel indoline-2-one derivative, induces systemic cartilage hyperplasia in rats, Eur J. Pharmacol. (2001) 418(3):225-30). Although the new generation of antirheumatic drugs have higher specificity to their molecular target, most of them do not have specificity to the diseased tissue, which lead to various side effects that limit their clinical application. Well-known side effects of NSAIDs include indigestion, stomach bleeding, liver and kidney damage, ringing in ears (tinnitus), fluid retention, and high blood pressure (Santana-Sahagun and Weisman, Nonsteroidal Anti-inflammatory Drugs, In Ruddy et al. (ed.) Kelley's Textbook of Rheumatology, 6^{th} Ed. W.B. Saunders Company, St. Louis, 1997, pp 799-822). Well known side effects of corticosteroids include bruising, thinning of bones, cataracts, weight gain, redistribution of fat, diabetes and high blood pressure (Stein and Pincus, Glucocorticoids, In Ruddy et al. (ed.) Kelley's Textbook of Rheumatology, 6^{th} Ed. W.B. Saunders Company, St. Louis, 1997, pp 823-840). Some DMARDs are immunosuppressants and usually lead to serious side effects, such as increased susceptibility to infection (Smolen and Steiner, Therapeutic strategies for rheumatoid arthritis, Nature Review Drug Discovery (2003) 2:473-488). The recent withdrawal of VIOXX® (COX-2 inhibitor, Merck) is a good example of the tremendous impact that side effects can have on an otherwise effective drug.

The ubiquitous in vivo distribution of receptors utilized by most of the antirheumatic drugs is a leading cause of their side effects. Therapeutic delivery systems, which could specifically deliver anti-arthritis drugs to the diseased tissue of RA patients, may avoid many of the side effects that are manifested in other tissues while achieving much greater clinical therapeutic efficacy.

The application of water-soluble polymers as a drug carrier for effective delivery of the drug to the desired sites (macromolecular therapy) has been extensively studied for the past two decades in the treatment of solid tumors (Kopecek et al., HPMA copolymer-anticancer drug conjugates: design, activity, and mechanism of action, Eur. J. Pharm. Biopharm. (2000) 50:61-81; Kopecek and H. Bazilova, Poly[N-(2-hydroxypropyl)methacrylamide], I. Radical polymerization and copolymerization, Eur. Polym. J. (1973) 9:7-14; Omelyanenko et al., Targetable HPMA copolymer-adriamycin conjugates, Recognition, internalization, and subcellular fate, J Controlled Release (1998) 53:25-37). Because of the “leaky” vasculature and poorly developed lymphatic system, extravasated macromolecules can be efficiently accumulated in the solid tumor. This phenomenon is termed tumor-selective “enhanced permeability and retention” (EPR) and has been used successfully to target anti-cancer drugs to solid tumors (Seymour, L. W., Passive tumor targeting of soluble macromoleculaes and drug conjugates, Critical Reviews In Therapeutic Drug Carrier Systems (1992) 9:135-187).

Studies using micro-particular carriers, such as liposomes for the delivery of anti-arthritic agents to a RA joint indicate some promising results in an animal model of arthritis (Metselaar et al., Complete remission of experimental arthritis by joint targeting of glucocorticoids with long-circulating liposomes, Arthritis Rheum. (2003) 48:2059-2066; see also Wunder et al., Albumin-based drug delivery as novel therapeutic approach for rheumatoid arthritis, J. Immunol. (2003) 170(9):4793-801; Timofeevski et al., Anti-inflammatory and antishock water-soluble polyesters of glucocorticoids with low level systemic toxicity, Pharm Res. (1996) 13(3):476-80). But the hepatotropism of the liposome may be problematic due to secondary livery toxicity. Therefore, there exists a need in the art for an effective drug delivery system that specifically targets RA joints.

### SUMMARY OF THE INVENTION

The invention relates to water-soluble polymeric delivery systems. In one embodiment, the delivery system is used for delivery of drugs to the diseased sites for the treatment of rheumatoid arthritis and other inflammatory diseases. In another embodiment, the delivery system is used for delivery of imaging agents to the diseased sites for non-invasive imaging, evaluation and diagnosis of rheumatoid arthritis and other inflammatory diseases.

In an exemplary embodiment, the invention provides water-soluble delivery systems for the delivery of anti-inflammatory therapeutic agents selected from the group consisting of proteins, peptides, lipoxins, resolvins, protectins, NSAIDs, DMARDs, glucocorticoids, methotrexate, sulfasalazine, chloriquine, gold, gold salt, copper, copper salt, penicillamine, D-penicillamine, cyclosporine, etc. and mixtures thereof, such drugs are well-known to those of skill in the art (The Pharmacological Basis of Therapeutics, 10^{th} ed, Gilman et al., eds., McGraw-Hill Press (2001); Remington's Pharmaceutical Science's, 18^{th} ed. Easton: Mack Publishing Co. (1990); The Resolution of Inflammation, Rossi et al., eds., Birkhauser Verlag AG (2008)).

In another exemplary embodiment, the invention provides a water-soluble polymeric delivery system for delivery of imaging agents, which are useful for non-invasive imaging and evaluation of arthritic joints and other inflammatory diseased organs or tissues. The imaging agents may be selected from any of the known compounds, for example, compounds useful for optical imaging, magnetic resonance imaging (MRI), positron emission tomography (PET), computerized tomography (CT) or gamma-scintigraphy imaging, etc. and mixtures thereof, such agents are well-known to those of skill in the art.

In another exemplary embodiment, the invention provides a water-soluble polymeric delivery system for delivery of a combination of imaging agents and anti-inflammatory therapeutic agents. In another exemplary embodiment, the invention provides a method of treating an inflammatory disease and monitoring the progress of the treatment. In another exemplary embodiment, the invention provides a method of screening anti-inflammatory therapeutic agents, wherein the anti-inflammatory agent is attached to a water-soluble polymeric delivery system of the invention and administered to a subject, the effect of the therapeutic agent is monitored, for example, using an imaging agent, and an effective therapeutic agent is identified. In another embodiment, the invention provides a method of screening anti-inflammatory therapeutic agents, wherein the anti-inflammatory agent is administered to a subject, the effect of the therapeutic agent is monitored, for example, using an imaging agent attached to a water-soluble polymeric delivery system of the invention, and an effective therapeutic agent is identified. Optionally, an imaging agent may be co-administered for the purpose of monitoring and/or screening the activity of the anti-inflammatory agent. Optionally, a targeting moiety or moieties may be used in the method of screening. The polymeric imaging agents of the instant invention can also be used for early detection and diagnosis of inflammatory disease.

In another exemplary embodiment, the inflammatory disease is rheumatoid arthritis, osteoarthritis (OA), temporomandibular joint syndrome (TMJ), inflamed nerve root, Crohn's disease, chronic obstructive pulmonary disease, psoriasis diseases, asthma, colitis, multiple sclerosis, systemic lupus erythematosus, atherosclerosis and/or the like.

In another exemplary embodiment, the invention relates to drug delivery systems comprising a water-soluble polymer backbone, optionally, a targeting moiety or moieties, and a therapeutic agent or agents, and/or an imaging agent. The linkage (or linkages) between the targeting moiety (or moieties) and the polymer backbone is non-degradable or degradable under physiological conditions. The linkage (or linkages) between the therapeutic agent (or agents) and the polymer backbone is non-degradable or degradable under physiological conditions.

In another exemplary embodiment, the invention relates to delivery systems for imaging agents comprising a water-soluble polymer backbone, optionally, a targeting moiety or moieties, and an imaging agent or agents. The linkage (or linkages) between the targeting moiety (or moieties) and the polymer backbone is non-degradable or degradable under physiological conditions. The linkage (or linkages) between the imaging agent (or agents) and the polymer backbone is non-degradable or degradable under physiological conditions.

In yet another exemplary embodiment, the invention provides a method of manufacturing a pharmaceutical composition and/or medicament comprising one or more delivery systems of the invention for the treatment of rheumatoid arthritis, osteoarthritis, temprormandibular joint syndrome, inflamed nerve root, Crohn's disease, chronic obstructive pulmonary disease, psoriasis diseases, asthma, colitis, multiple sclerosis, systemic lupus erythematosus, atherosclerosis and/or the like.

As will be apparent to a person of ordinary skill in the art based on the invention described herein, the invention provides the advantage of incorporating multiple therapeutic agents, targeting moieties, spacers, bio-assays labels, and/or imaging agents, which may include a plurality of different chemical species from one or more of these groups. Therefore, in yet another exemplary embodiment the therapeutic agents, targeting moieties, spacers, bio-assays labels, and/or imaging agents may consist of any number or combination of different species, having the same or different effects.

In accordance with another aspect of the instant invention, methods of treating an inflammatory disease in a subject in need thereof are provided. In a particular embodiment, the method comprises administering to the patient a composition comprising at least one N-(2-hydroxypropyl)methacrylamide (HPMA) copolymer and at least one pharmaceutically acceptable carrier. In another embodiment, the HPMA copolymer comprises an anti-inflammatory therapeutic agent conjugated to the copolymer via a pH-sensitive linker, such as a hydrazone containing linker. The copolymer may also comprise at least one imaging agent. In another embodiment, the method further comprises the administration of at least one additional anti-inflammatory therapeutic agent.

In accordance with another aspect, monomers for generating the copolymers of the instant invention are provided. In a particular embodiment, the monomer is linked to at least one therapeutic agent and/or imaging agent. The instant invention also provides methods of synthesizing a copolymer with the monomers of the instant invention.

Methods of imaging an inflammatory disease in a subject are also provided. In a particular embodiment, the method comprises administering to the patient a composition comprising at least one N-(2-hydroxypropyl)methacrylamide (HPMA) copolymer and at least one pharmaceutically acceptable carrier, wherein said HPMA copolymer comprises at least one imaging agent conjugated to the copolymer via a linker.

### BRIEF DESCRIPTION OF THE DRAWING

FIG. 1 illustrates the chemical structure of an exemplary polymeric delivery system for MRI contrast agent (1,4,7,10-tetraazacyclododecane-1,4,7,10-tetra (acetic acid) (DOTA)-Gd³⁺), abbreviated as P-DOTA-Gd³⁺, for the imaging of arthritic joints and evaluation of the severity of the disease.

FIG. 2 shows the histology of the ankle and knee joints from the adjuvant-induced arthritis (AIA) rats that were also imaged by MRI. FIG. 2A is a low power micrograph of the ankle and foot bones. Extensive swelling and inflammation is evident in the soft tissues (*) surrounding the foot bones. T=tibia. FIG. 2B shows the tarsal joint illustrating inflamed synovium (synovitis), extensive inflammatory infiltration (*) and cartilage and bone destruction. B=bone, A=articular cartilages. FIG. 2C is a higher power micrograph of the inflamed synovium. A=articular cartilage. FIG. 2D shows extensive bony destruction with inflammatory infiltration (*) in a tarsal (ankle) bone. Bone surfaces are lining with large active osteoclasts (arrows). FIG. 2E illustrates several blood vessels in an inflamed region of the ankle joint illustrating the inflammatory reaction around the vessels. The endothelial lining is thickened and vacuolated (arrows). FIG. 2F is a low power micrograph of the knee joint from this same animal. There joint is quite normal in appearance except for a small pocket of inflammation on the posterial aspect of the joint (arrow). This same area was contrasted when observed by MRI. T=tibia; F=femur.

FIG. 3 shows the MR images of the animals taken at different time points. The acquired images were post processed using the maximum intensity projection (MIP) algorithm. FIG. 3A shows AIA rat baseline; FIG. 3B shows AIA rat, 5 minutes post injection of P-DOTA-Gd³⁺; FIG. 3C shows AIA rat, 1 hour post injection of P-DOTA-Gd³⁺; FIG. 3D shows AIA rat, 2 hours post injection of P-DOTA-Gd³⁺; FIG. 3E shows AIA rat, 3 hours post injection of P-DOTA-Gd³⁺; FIG. 3F shows AIA rat, 8 hours post injection of P-DOTA-Gd³⁺; FIG. 3G shows AIA rat, 32 hours post injection of P-DOTA-Gd³⁺; FIG. 3H shows AIA rat, 48 hours post injection of P-DOTA-Gd³⁺; FIG. 3I shows healthy rat, baseline; FIG. 3J shows healthy rat, 5 minutes post injection of P-DOTA-Gd³⁺; FIG. 3K shows healthy rat, 1 hour post injection of P-DOTA-Gd³⁺; FIG. 3L shows healthy rat, 2 hours post injection of P-DOTA-Gd³⁺; FIG. 3M shows healthy rat, 8 hours post injection of P-DOTA-Gd³⁺; FIG. 3N shows healthy rat, 48 hours post injection of P-DOTA-Gd³⁺; FIG. 3O shows AIA rat, 5 minutes post injection of OMNISCAN (or gadodiamide is the injectable formulation of the gadolinium complex of diethylenetriamine pentaacetic acid bismethylamide); FIG. 3P shows AIA rat, 2 hours post injection of OMNISCAN; FIG. 3Q shows AIA rat, 8 hours post injection of OMNISCAN; FIG. 3R shows AIA rat, 32 hours post injection of OMNISCAN; FIG. 3S shows AIA rat, 48 hours post injection of OMNISCAN; FIG. 3T shows healthy rat, 5 minutes post injection of OMNISCAN; FIG. 3U shows healthy rat, 1 hour post injection of OMNISCAN; FIG. 3V shows healthy rat, 2 hours post injection of OMNISCAN; FIG. 3W shows healthy rat, 8 hours post injection of OMNISCAN; FIG. 3X shows healthy rat, 48 hours post injection of OMNISCAN.

FIG. 4 illustrates single-plane MR imaging of AIA rats injected with P-DOTA-Gd³⁺. FIG. 4A shows baseline MR image of AIA rat; FIG. 4B shows an MR image of AIA rat's left ankle and paw, 2 hours post injection; FIG. 4C shows an MR image of AIA rat's left ankle and paw, 8 hours post injection; FIG. 4D shows an MR image of AIA rat's left knee joint, 8 hours post injection.

FIG. 5 illustrates the general structure of the water-soluble polymeric delivery system. The average mol percentage of targeting moieties (T) per polymer chain may range anywhere from 0% to about 50%, preferably from 0% to 30%. The average mol percentage of therapeutic agents (D) or mixture of both per polymer chain may range anywhere from 1% to about 90%. The average mol percentage of bio-assay label or imaging agents (L) per polymer chain may range anywhere from 0% to about 50%. The spacer S₁ and S₂ can be covalent or physical bonds or linkages, such as peptides or other complex chemical structures, which may or may not be cleaved upon stimulus, such as change of pH, specific enzyme activity (e.g., cathepsins (e.g., cathepsin K), MMPs, etc.), presence or absence of oxygen, etc. under physiological condition. The spacer S₃ illustrates a non-degradable, under physiological condition, covalent or physical bond or linkage. The optional biodegradable cross-linkage (C) can be covalent or physical bonds or linkages, such as peptides or other complex chemical structures, which may be cleaved upon stimulus, such as a change of pH, specific enzyme activity (e.g., cathepsins (e.g., cathepsin K), MMPs, etc.), presence or absence of oxygen, etc., under physiological conditions.

FIG. 6 illustrates the chemical structure of a exemplary polymeric drug delivery system, with dexmethasone as an example of a therapeutic agent, for the treatment of rheumatoid arthritis and other inflammatory disorders. The polymeric prodrug is abbreviated as P-Dex (conjugate of dexamethasone to copolymer of HPMA, MA-GG-NHNH₂ (N-methacryloyl glycylglycyl hydrazine) and MA-FITC (fluorescein isothiocyanate) via hydrazone bond).

FIG. 7 shows the superior therapeutic effect of P-Dex over dexamethasone sodium phosphate (Dex) in reducing the size of the inflamed arthritic joints during treatment.

FIG. 8 shows the superior therapeutic effect of P-Dex over dexamethasone sodium phosphate (Dex) in the bone mineral density (BMD) of the inflamed arthritic joints during treatment. The results were obtained by dual x-ray absorptiometry (DEXA).

FIG. 9 shows the superior therapeutic effect of P-Dex over dexamethasone sodium phosphate (Dex) in reducing erosion of the bone surface of inflamed arthritic joints during treatment. The results were obtained by histomorphometry using a Bioquant image analysis system.

FIG. 10 shows the superior therapeutic effect of P-Dex over dexamethasone sodium phosphate (Dex) by histological observation of the inflamed arthritic joints during treatment.

FIG. 11 demonstrates the in vitro Dex release from P-Dex at pH=5.0 and 7.4. Each sample was measured 3 times. The mean values and standard deviation were calculated with Microsoft Excel. For the linear regression, R²>0.99.

FIG. 12 demonstrates the change of right ankle joint diameter of four animal groups (P-Dex, Dex, saline, and healthy) during the entire experiment.

FIG. 13 is a graph showing the change of articular index (AI) score of four animal groups (P-Dex, Dex, saline, and healthy) during the entire experiment.

FIG. 14 shows the endpoint bone mineral density (BMD) and representative pDEXA images of the right ankle joints of four animal groups (P-Dex, Dex, saline, and healthy). One-way ANOVA analysis, P<0.01.

FIG. 15 is a graph of the histological evaluation of the ankle joints from four animal groups (P-Dex, Dex, saline, and healthy). One-way ANOVA analysis, P<0.0001.

FIGS. 16A-16D provide representative histology pictures of the ankle joints from four animal groups. FIG. 16A: P-Dex; FIG. 16B: Healthy; FIG. 16C: Free Dex; FIG. 16D: Saline. Synovial cell lining and villous hyperplasia (**), bone destruction (single arrow) and cartilage damage (double arrow) are clearly evident in free Dex and saline groups. Tib=Tibia, Ast=astrogalus. Bar=0.5 mm.

FIG. 17A provides scheme 1 for the synthesis of MA-Gly-Gly-NHN=Dex. FIG. 17B provides scheme 2 for the synthesis of HPMA copolymer-Dex conjugate (P-Dex) via RAFT copolymerization. FIG. 17C provides scheme 3 for the structures syn/anti diastereomers of MA-Gly-Gly-NHN=Dex.

FIG. 18 provides a scheme for the synthesis of poly(HPMA-co-APMA)-IRDye 800 CW conjugate. FIG. 18B provides a scheme for the synthesis of linear multifunctional PEG-IRDye 800 CW conjugate using click chemistry.

FIG. 19 provides images of optical imaging of a healthy rat and adjuvant induced arthritis (AIA) rat using HPMA copolymer-IRDye conjugate as the contrast agent.

FIG. 20 provides structures of monomers.

### DETAILED DESCRIPTION OF THE INVENTION

Throughout the description of the invention and the claims, and following convention, the “singular” includes the “plural”; for example, a therapeutic agent and/or a targeting moiety, means at least one such therapeutic agent or targeting moiety, unless indicated otherwise.

To demonstrate the principle of the invention, conventional visual examination with Evans blue dye (EB) injection and magnetic resonance imaging (MRI) techniques (Brown and Semelka, Principles of Magnetic Resonance Imaging, In Brown and Semelka (ed.) MRI: Basic Principles and Applications, 3^{rd} Ed. Wiley-Liss, New York, 2003, pp 27-42; Demsar et al., Mapping abnormal synovial vascular permeability in temporomandibular joint arthritis in the rabbit using MRI, Br. J. Rheumatol. (1996) 35(Suppl 3):23-25; Jacobson et al., A new spin on an old model: in vivo evaluation of disease progression by magnetic resonance imaging with respect to standard inflammatory parameters and histopathology in the adjuvant arthritic rat, Arthritis Rheum. (1999) 42 (10):2060-73) were used to follow the in vivo fate of macromolecules on an established AIA rat model. Additionally, histological examination confirmed the presence of disease in specific anatomical locations where the macromolecular delivery system is identified with MRI technique.

EB is a commonly used agent to assess vascular permeability and integrity (Jacobs et al., Blood flow and vascular permeability during motor dysfunction in a rabbit model of spinal cord ischemia, Stroke (1992) 23:367-373; Kushner and Somerville, Permeability of human synovial membrane to plasma proteins, Relationship to molecular size and inflammation, Arthritis and Rheumatism (1971) 14:560-570). It is a dye-carrying multiple charges and aromatic structures, which forms a strong complex with plasma albumin. Injection of the dye had been successfully used to establish the concept of macromolecular therapy for the treatment of solid tumors (Matsumura and Maeda, A new concept for macromolecular therapeutics in cancer chemotherapy: mechanism of tumoritropic accumulation of proteins and the antitumor agent smancs, Cancer Res. (1986) 46:6387-6392). In this experiment, the EB dye technique was used in AIA rats to visually assess the accumulation of plasma albumin in inflamed joints. The hind paw of the AIA rats, where the most severe inflammation was evident, readily incorporated the dye compared with that observed in the healthy rats. This observation confirmed that there was indeed a much greater concentration of plasma albumin in the inflamed joints of the AIA rat model.

Although the results with EB are significant, it is noted that the dye is not covalently bound to albumin and some dye transfers nonspecifically to other tissues. For example, a slight blue staining was evident in some organs, including the liver and heart.

Magnetic resonance imaging (MRI) is a noninvasive method of mapping the internal structure of the body. It employs radiofrequency (RF) radiation in the presence of carefully controlled magnetic fields in order to produce high quality cross-sectional images of the body in any plane. It portrays the distribution of hydrogen nuclei and parameters relating to their motion in water and lipids. Introduction of paramagnetic contrast agents would shorten T₁ (the longitudinal relaxation time) of the hydrogen nuclei in tissues, which in turn will increase the MR signal intensity thereof (Paley et al., Magnetic resonance imaging: basic principles, In Grainger et al. (ed.) Grainger & Allison's Diagnostic Radiology: A Textbook of Medical Imaging, 4^{th} Ed. Churchill Livingstone, Inc. London, 2001, pp 101-136). Therefore, to further support the results, magnetic resonance imaging (MRI) was used to track the DOTA-Gd³⁺ labeled macromolecules injected in AIA rats.

It is well understood that obtaining a higher MR contrast signal intensity in the MR images represents the existence of a higher concentration of the paramagnetic contrast agents in the tissue. The analysis of the macromolecular contrast agent enhanced MR images of the rats provides important information about the pharmacokinetics profile and biodistribution of the water-soluble polymeric delivery systems described in this invention. In addition, such imaging agents will also enhance the sensitivity and anatomical resolution of the resulting images of a subject (preferably a mammal, such as a human), an animal (including, an animal model for a particular disease, dog, cat, horse or livestock), or part (e.g., a tissue or structure) of the subject or animal.

Conjugation of a low molecular weight paramagnetic contrast agent, DOTA-Gd³⁺ complex to HPMA copolymer enabled the non-invasive monitoring of the fate of the injected polymer in rats with MR scanner. This approach of labeling the polymer with a MRI contrast agent is similar to labeling the polymers with fluorochromes to permit localization in organs, tissues and cells. Alternatively, this approach to imaging may also be used with other imaging agents for optical imaging, PET, CT and gamma-scintigraphy for the purposes of non-invasive imaging, diagnosis (particularly early diagnosis), evaluation of the diseased tissues and organs and detection of molecular targets in the tissues or organs of interest, etc.

Therefore, a macromolecular magnetic resonance imaging (MRI) contrast agent based on N-(2-hydroxypropyl)methacrylamide (HPMA) copolymer has been synthesized to illustrate the invention. After systematic administration of the contrast agent in an adjuvant-induced arthritis (AIA) rat model, contrast enhanced MR images were taken, which show the distribution of the polymer at different time points. Correlating the MR results with additional visual and histopathological results from the AIA rats, demonstrates the preferential deposition and retention of macromolecules in the inflamed joints. Thus, demonstrating the effectiveness of using macromolecular therapy for the treatment of rheumatoid arthritis. In addition, these results demonstrate the feasibility of using macromolecular imaging agents for imaging, diagnosis, and evaluation of arthritic joints.

The invention includes polymeric delivery systems for the delivery of drugs, such as anti-inflammatory and/or inflammation resolution drugs (e.g., lipoxins, resolvins, and protectins).

The invention includes polymeric delivery systems for the delivery of imaging agents, such as chemical compounds used as enhancing agents in MRI (for example, DOTA-Gd³⁺, DTPA-Gd³+(gadolinium complex with diethylenetriamine pentaacetic acid), etc.), PET (for example, compounds labeled or complexed with ¹¹C, ¹³N, ¹⁵O, ¹⁸F, ⁶⁴Cu, ⁶⁸Ga, ⁸²Rb, etc., such as ¹⁸F-FDG (fluorodeoxyglucose)), CT (for example, iodine or barium containing compound, such as 2,3,5-triiodobenzoic acid) and gamma-scintigraphy (for example, compounds complexed with ⁹⁹Tc, ¹¹¹In, ¹¹³In, and ¹⁵³Sm, etc.) and optical imaging (e.g., near infrared dyes, chromophore, fluorescent compound, fluorescein, and the like).

### MRI Procedure

MR images of the animals were acquired on a 1.5 T Signa LX imaging system (General Electric Medical Systems, Milwaukee, Wis.), using a phased-array coil. Images were acquired using a 3D single slab IR prepped FSPGR sequence in the coronal plane. The common imaging parameters were TR=13.4 ms, TE=2.2 ms, TI=300 ms, 25° flip angle, 512×256 in-plane acquisition matrix, 20×10 cm² field-of-view (FOV), 64 slices per slab, 1.0 mm thick slices with 2×interpolation to 0.5 mm.

Synthesis of Poly(HPMA-co-APMA-co-MA-FITC) (copolymer of HPMA, APMA and MA-FITC)

N-(2-hydroxypropyl)methacrylamide (HPMA; 1 g, 7 mmol), N-(3-Aminopropyl)methacrylamide hydrochloride (APMA; 0.14 g, 0.78 mmol), N-methacryloylaminopropyl fluorescein thiourea (MA-FITC; 0.043 g, 7.8 mmol), 2,2′-azobisisobutyronitrile (AIBN; 0.057 g, 0.35 mmol) and mercaptopropionic acid (MPA; 0.001 mL, 1 mmol) were dissolved in methanol (10 mL), placed in an ampoule and purged with N₂ for 5 minutes. The ampoule was flame-sealed and maintained at 50° C. for 24 hours. The polymer was isolated by precipitation of the resulting solution into acetone and was reprecipitated twice. After the polymer was dried in desiccator (over NaOH), the final yield was determined as 0.9 g. The content of free amino groups in the copolymer was determined as 7.7×10⁴ mol/g using the ninhydrin assay (Moore and Stein, A modified ninhydrin reagent for the photometric determination of amino acids and related compounds, J. Biol. Chem. (1954) 211:907-913).

Synthesis of P-DOTA (Conjugation Product of poly(HPMA-co-APMA-co-MA-FITC) and DOTA-NHS ester)

Poly(HPMA-co-APMA-co-MA-FITC) (170 mg, [NH₂]=1.33×10⁻⁴ mol), DOTA-NHS ester (1,4,7,10-tetraazacyclododecane-1,4,7-tris(acetic acid)-10-acetic acid mono (N-hydroxysuccinimidyl ester; 100 mg, 2×10⁻⁴ mol) and diisopropylethyl amine (DIPEA, 160 mL, 9.33×10⁻⁴ mol, distilled from ninhydrin) were mixed in DMF (1.5 mL, distilled from P₂O₅) and stirred overnight. The conjugate was precipitated into ether and dried in vacuum. The product was further purified on LH-20 column, dialyzed (molecular weight cutoff size is 6-8 kDa) and lyophilized to obtain 190 mg of final product. The residue free NH₂ group was determined with ninhydrin assay and the content of DOTA in the product was calculated as 7.5×10⁻⁴ mol/g.

Synthesis and Purification of Macromolecular MRI Contrast Agent P-DOTA-Gd³⁺ (purified complex of P-DOTA and Gd³⁺)

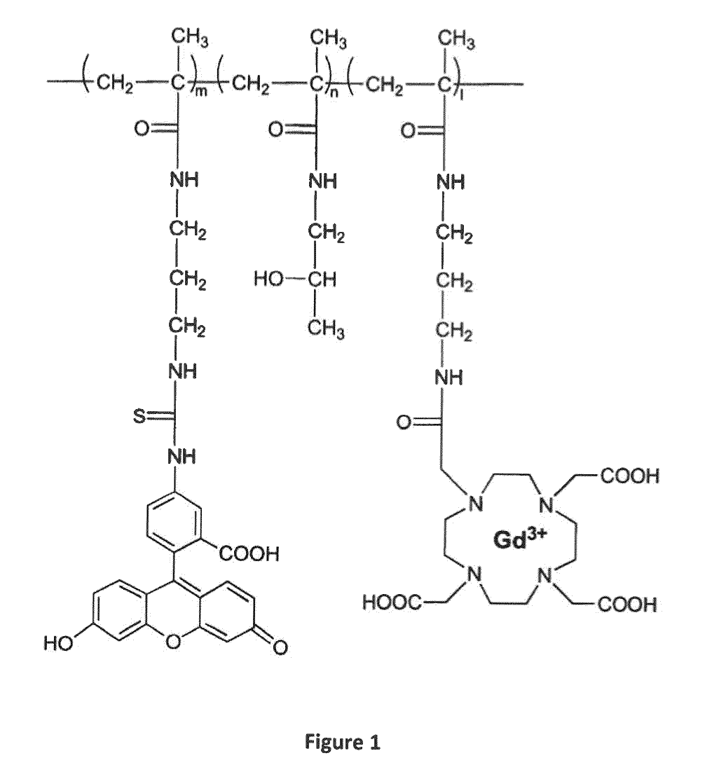

P-DOTA (100 mg, [DOTA]=6.9×10⁻⁵ mol) and GdCl₃.6H₂O (38 mg, 1.04×10⁻⁴ mol) were dissolved in 2 mL deionized H₂O. The pH of the solution was maintained at 5.0-5.5 over night by gradual addition of NaOH (1 N) solution. EDTA disodium salt (38 mg, 1.04×10⁴ mol) was then added into the solution to chelate the excess Gd³⁺. After stirring for 30 minutes, the milky solution was purified with Sephadex G-25 column to remove the EDTA-chelated Gd³⁺ and other unreacted low molecular weight compounds from the polymer conjugate. The conjugate was lyophilized to yield 115 mg P-DOTA-Gd³⁺. The gadolinium content was determined by Inductively Coupled Plasma Optical Emission Spectroscopy (ICP-OES) as 0.52 mmol/g. The Mw of the polymeric MRI contrast agent is determined fast protein liquid chromatography (FPLC) as 55 kDa with a polydispersity of 1.43. The T₁ relaxivity of the conjugate was determined as 10.4 mM⁻¹s⁻¹ per complexed Gd³⁺ using a B1 homogeneity corrected Look-Locker technique on the 1.5T GE NV/Cvi scanner with the LX 8.4 operating system at room temperature (RT) (Lu et al., Poly(1-glutamic acid) Gd(III)-DOTA conjugate with a degradable spacer for magnetic resonance imaging, Bioconjug Chem. (2003) 14:715-719). The chemical structure of the macromolecular MRI contrast agent is shown in FIG. 1.

### Synthesis of P-Dex

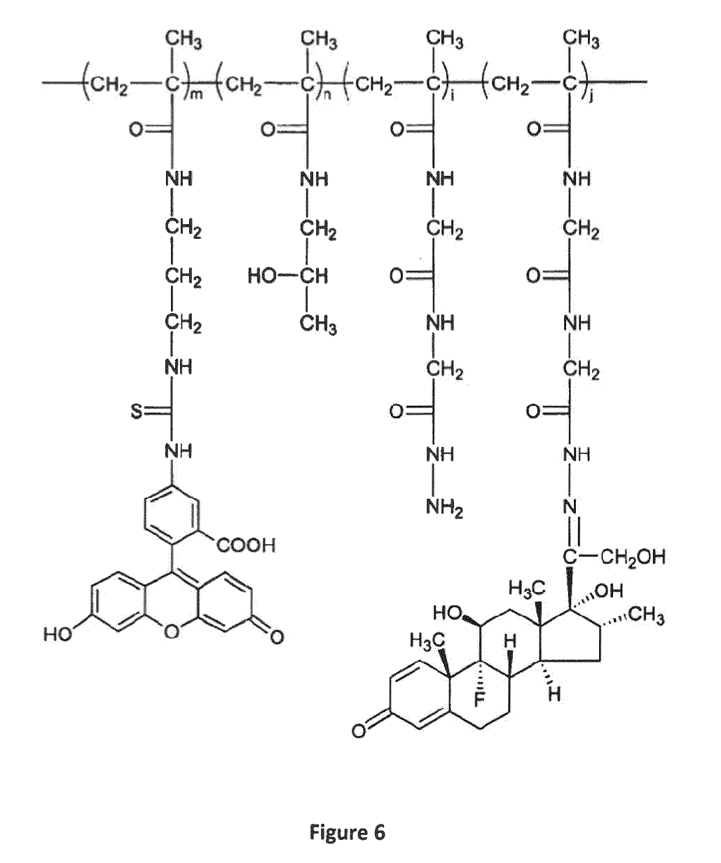

HPMA (1 g, 0.00698 mol), MA-GG-OH (0.156 g, 0.00078 mol), MA-FITC (0.02 g, 0.00004 mol) and AIBN (0.007 mg, 0.000043 mol) were dissolved in DMSO (1 mL) and MeOH (8 mL) mixture. The solution was transferred into an ampoule and purged with N₂ for 5 minutes and then polymerized at 50° C. for 24 hours. The polymer was then reprecipitated twice to yield about 1 g of copolymer. It was further activated with a large excess of hydroxy succinimide (HOSu) and then reacted with hydrazine. After reprecipitation, dexamethasone was conjugated to the copolymer in the presence of 1 drop of acetic acid in DMF. The conjugate was purified with LH-20 column and freeze-dried to obtain the final conjugate (structure shown in FIG. 6) with dexamethasone content of 49 mg/g (of conjugate).

### Adjuvant Induced Arthritis (AIA) Rat Model

Male Lewis rats (175-200 g) were obtained from Charles River Laboratories (Portage, Minn.) and allowed to acclimate for at least one week. To induce arthritis, Mycobacterium Tuberculosis H37Ra (1 mg) and N,N-dioctadecyl-N′,N′-bis(2-hydroxyethyl) propanediamine (LA; 5 mg; U.S. Pat. No. 4,034,040) were mixed in paraffin oil (100 μL), sonicated and s.c. injected into the base of the rat's tail (Bendele, A. M., Animal models of rheumatoid arthritis, J. Musculoskel. Neuron. Interact. (2001) 1:377-385). The rats were then randomized into 3 rats/group. The progression of the joint inflammation was followed by measuring the diameter of the ankle joint with calipers. Special care was given to the rats as the inflammation developed to ensure availability and access to water and food. The MRI contrast agents used for the study were injected directly into the jugular vein while the animal was anesthetized with Ketamine and Xylazine.

### Visualization of Plasma Albumin Accumulation in RA Joints

Evans blue dye (EB, 10 mg/kg in saline) was injected into healthy and AIA rats via the tail vein. The extravasation and accumulation of dye in the areas of joint inflammation could be visually observed as appearance of the blue pigment. Photographs of the ankle and paws were taken before and 8 hours after injection.

### Histology

At necropsy, the major organs and limbs were removed and fixed with 10% phosphate buffered formalin for 24 hours. The organs were then dehydrated and embedded in paraffin for routine histopathological analyses. The limbs were gradually dehydrated in ascending concentrations of ethanol and embedded in poly(methyl methacrylate). Sections of the entire joint, including the undecalcified bone, were cut with a low speed saw using diamond-wafering blades. The sections were mounted on plastic slides, ground to about 50 μM in thickness and surface stained using a Giemsa stain modified for plastic sections (Wang et al., Synthesis and evaluation of water-soluble polymeric bone-targeted drug delivery systems, Bioconjug. Chem. (2003) 14:853-859). The joints (knee, ankle, tarsals and metatarsals) from the same animals that were imaged by MRI were assessed for the presence of inflammation and tissue damage using the histology sections. A Bioquant histomorphometry system was used to measure the bone erosion surface.

### Bone Mineral Density

The bone mineral density (BMD) of the bones in the arthritic joints was measured by peripheral dual x-ray absorptiometry (PDXA, Norland Medical Systems) adapted for small animals. For this the intact hind limbs were used and the scan region included the ankle and foot bones. The coefficient of variation between measurements was less than 1%.

### Visual and Histological Examination of AIA Rats

The development of adjuvant-induced arthritis in the rat is well described in the literature (Bendele, A. M., Animal models of rheumatoid arthritis, J. Musculoskel. Neuron. Interact. (2001) 1:377-385), and briefly summarized here. After injection of the adjuvant, changes begin to become evident about 9 days later. This includes some inflammation around the eyes and enlarged and tender external genitalia. Inflammation and swelling of the front and hind limb ankle joints becomes evident at about 12 days after injection of the adjuvant.

At necropsy at 15 days post injection of the adjuvant, inflammation of the peritoneum (peritonitis) can be observed. Occasionally, inflammation of gastrointestinal (GI) tract and fluid retention in the peritoneal cavity are also detectable. Grossly, most of the vital organs appear to be normal except that the spleen is usually enlarged with visual evidence of inflammation.

However, under histopathological examination, all organs examined showed signs of chronic inflammation. The testicular tissue demonstrates inflammation in the membranes around the testis, with small granulomata in the epididymis being detected. The pericardial tissue demonstrates chronic inflammation, which easily could allow for buildup of fluid in the pericardial tissue. The renal tissue includes multifocal areas of granulomata formation in the cortical tissue with some inflammation over the capsule, particularly along potential serosal surface changes. The splenic tissue demonstrates multifocal areas of necrosis surrounded by neutrophils and epithelioid cells. Plasma cells and lymphocytes are responding around this process, which indicate a rather severe inflammatory response throughout the splenic tissue.

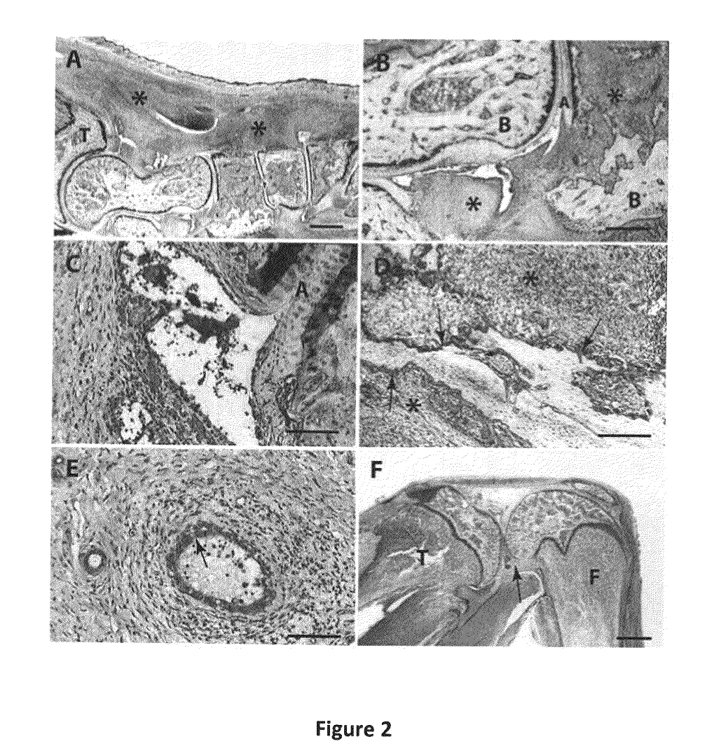

The histological images of the AIA rats' hind legs are presented in FIG. 2. At lower magnification, the swelling of the ankle joint region and paws of the AIA rats is evident (FIG. 2A). Extensive inflammation, synovitis, bone and cartilage destruction is evident (FIGS. 2B to 2D). Inflammatory cells are observed around the larger vessels (FIG. 2E). By contrast, the knee joints from the AIA rats are typically less affected by the inflammatory process (FIG. 2F).

Visual Examination of the AIA Rats after Evans Blue Injection

For this experiment, EB was injected into the rats 15 days after injection of the adjuvant. By this time, there is a robust inflammatory reaction evident in the ankle joints. After injection of EB, there was a gradual accumulation of blue color in the inflamed hind paw and front paws of the AIA rats with high density of the color located around the tarsus and carpus. Some deep blue spots were also observed on some digits of the paw. Photographs taken before and after injection of the EB dye confirm that the areas with dye accumulation correspond to those with marked inflammation. In the healthy control rats given EB, the dye was not localized to joint areas as observed in the AIA rats.

### MR Imaging

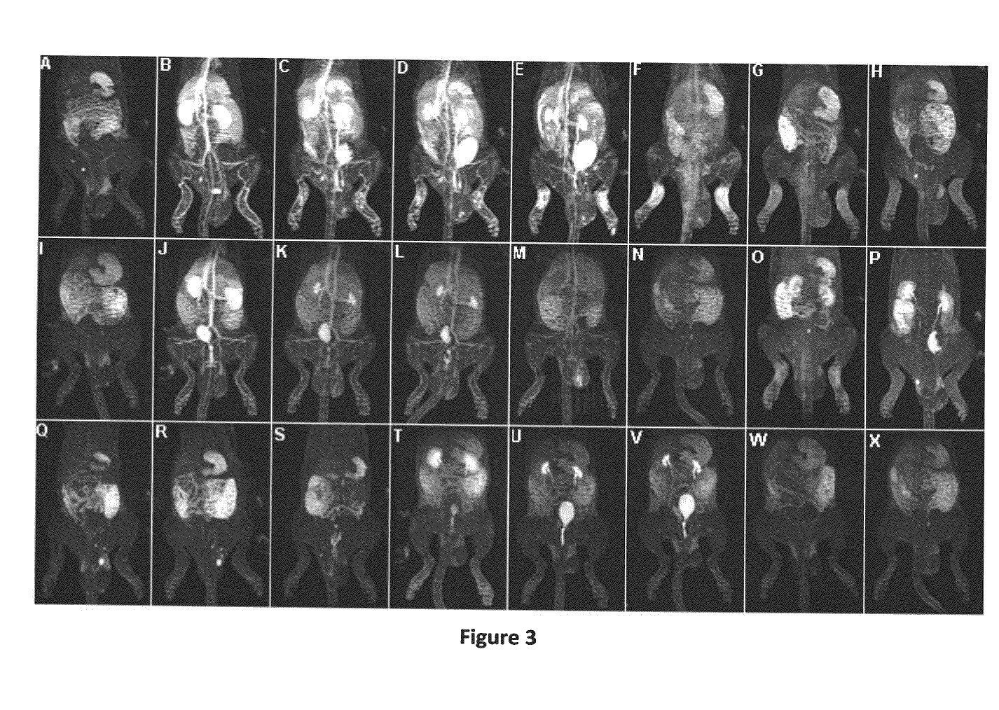

Imaging AIA rats with P-DOTA-Gd³⁺ as a contrast agent. Immediately prior to the injection of the P-DOTA-Gd³⁺ contrast agent, a baseline MRI scan was done. The animals were then injected with the contrast agent and MRI scans were performed at different time intervals. The acquired images were post processed using the maximum intensity projection (MIP) algorithm. The resulting MIP images of the animals are depicted chronologically in FIG. 3.

As shown in the baseline image (FIG. 3A) before contrast injection, the intestine and stomach of the animal are clearly visible likely due to fluid retention. Several irregular spots are also observed in the lower abdomen, which can be attributed to i.p. injection site(s) of anesthetic agents. An area in the scrotum, adjacent to the testes in the anatomical region of the epididymus and associated tissue also shows a diffuse MR signal, perhaps due to its fatty content or accumulation of fluid. The bright spot at the right sciatic region may represent the fluid retention in an inconsistent lymph node called Ic. Ischiadicum, which is also evident in some of the subsequent images from this animal. No significant MR signal was observed at the hind limbs. The ankle joints were, however, clearly enlarged in the latent image when compared with similar images of the controls.

At five minutes after the injection of the macromolecular contrast agent, there was substantial MR signal in the kidneys (FIG. 3B). A detailed examination of the single-plane 2-dimensional images indicates that at this time most of the contrast agent is in the kidney cortex with little in the medulla. Because of the overall increase of the image contrast after the injection, the bladder became evident as a negative image (dark) as the oval shaped structure at lower left abdominal area. Increased contrast is also observed in the liver, spleen and bone marrow. The major blood vessels are clearly defined while the lesser vessels are not as apparent, probably due to the limited imaging resolution (about 0.5 mm) with the 1.5 T MRI scanner. However, the vessels appear larger, perhaps dilated, than those observed in the healthy controls. Except for some uptake in the bone marrow, little significant contrast signal was evident at this time in the inflamed ankle region.

In the MR images (FIG. 3C) of the AIA rats acquired 1 hour post injection, the signal in the cortex of kidneys was greatly reduced compared with the earlier (5 minute) time. However, now most of the contrast appears to be concentrated in the kidney medulla and pelvis. Both ureters contain contrast material and a substantial signal is now evident in the urinary bladder. There appeared to be slight decrease in the MR contrast signal in the liver, spleen and blood vasculature. Interestingly, several “hot spots” start to appear around the tarsus, where the most severe inflammation occurs in this animal model.

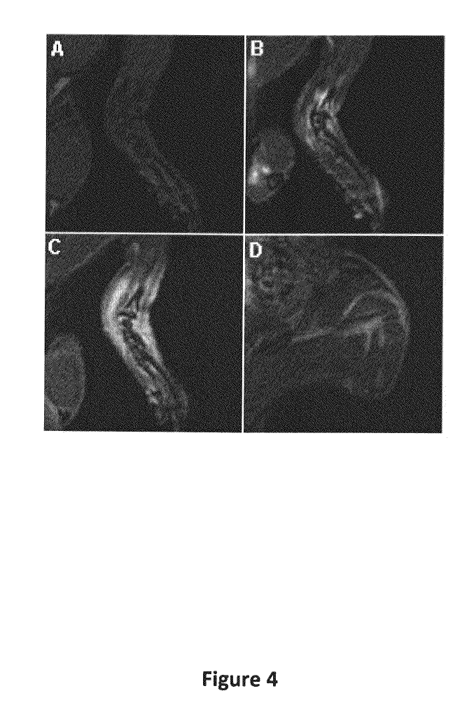

From the MR images acquired 2 hours (FIG. 3D) and 3 hours (FIG. 3E) post injection of the macromolecular contrast agent, a gradual reduction of MR contrast signal was evident in the kidney (cortex and medulla), liver, spleen and vasculature. There was, however, an accumulation of the contrast material in the urinary bladder. The “hot spots” detected around the tarsus at the 1 hour scan continue to expand and increase in contrast in the 2 hours (FIG. 4B) and 3 hours images.

When the rats were scanned again at 8 hours post injection, the MR images (FIG. 3F) acquired show greatly reduced MR signal in all the vital organs and blood vessels with essentially an undetectable bladder, even though the overall body signal remains slightly greater than that of the baseline images. Surprisingly, however, the MR contrast signal is substantially increased in the ankle joint and metatarsal region (FIG. 4C). The initial “hot spots” disappear and the MR contrast signal is more evenly distributed around the joint tissue. Also observed is some contrast signal in the posterior knee joints, but with a much less intensity and size (FIG. 4D).

Subsequently, the animals were again scanned at 32 hours and 48 hours post injection, respectively (FIGS. 3G and 3H). The overall contrast enhancement of MR signal continued to decline from that observed in the 8 hour images. However, the decrease in image contrast in the ankle joint tissue appeared to be much slower than observed in other tissues and organs. Even after 48 hours, the enhancing effect of the injected macromolecular contrast agents is still visible in the hind ankle and paw tissue.

Imaging healthy rats with P-DOTA-Gd³⁺ as a contrast agent. In the MR images (FIG. 3J) taken 5 minutes after the injection of macromolecular contrast agent, the kidneys of the healthy animals showed extremely strong contrast signal. The single-plane 2-D images indicate that the MR contrast resides in both the cortex and medulla. Both side ureters are partially visible. The urinary bladder is filled with a significant amount of contrast medium. Liver, spleen and bone marrow were visible in the image when compared with the baseline image. The major blood vessels, including the abdominal aorta and inferior vena cava were also highlighted. The arrangement and appearance of these vessels appears to be normal. No contrast signal was detected outside of the large vessels in the hind paws.

In the MR images taken at 1 hour (FIG. 3K) and 2 hours (FIG. 3L) post injection, little contrast media remains in the kidney cortex and medulla, but some contrast signal remained in the kidney pelvis and bladder. The contrast enhancement of the vasculature was slightly reduced compared with that observed at 5 minutes after injection. At 8 hours (FIG. 3M) after injection, the contrast media was completely cleared from the urinary tract. At this time, some of the large vessels were still evident, though less so than at earlier times. The images taken at 48 hours (FIG. 3N) after injection replicate the baseline images with no detectable contrast enhancement. As expected, all MR images taken at different times post injection did not show contrast enhancement in the hind-limb joints of the animal. Imaging AIA and healthy rats with OMNISCAN as contrast agent.

The images acquired with the MR enhancement of a low molecular weight paramagnetic contrast agent OMNISCAN (gadolinium complex of diethylenetriamine pentaacetic acid bismethylamide) were obtained similarly as those injected with P-DOTA-Gd³⁺.

From the image sequence presented in FIG. 3 (30 to 3×), a very fast overall tissue contrast enhancement at 5 minutes post injection was observed in both healthy and AIA rats (FIGS. 3O & 3T). However, the contrast enhancement quickly declined, accompanied by a rapid renal clearance of the contrast medium. At 8 hours (FIGS. 3Q & 3W), the enhancement was basically gone. Interestingly, the 5-minute images (FIG. 3O) of the AIA rats reveal significant contrast enhancement at the inflamed ankle joints, which had cleared at the 2 hours scan (FIG. 3P). However, no such observation was found in the healthy rats. Basically, no blood vasculature contrast enhancement could be observed in all OMNISCAN enhanced MR images.

As shown in FIG. 3, all vital organs in AIA rats showed greater uptake of P-DOTA-Gd³⁺ than the healthy rats. In addition, the clearance of the contrast agent in these organs was slower than those in healthy rats, especially in the kidneys. These observations are consistent with the histological findings that all organs in AIA rats, including heart, liver, lung, kidney and spleen had some granulomatous chronic inflammation. The vasculature in such inflamed tissues is often more porous, permitting a greater extravasation of macromolecules to the interstitial tissue. These may lead to organ dysfunction, such as the delayed renal clearance of the polymer contrast agent compared to healthy rats. However, the major clearance of P-DOTA-Gd³⁺ from these organs was completed within a few hours (<8 hours) in the AIA rats. When compared with normal rats, the major blood vessels appear to be dilated in the AIA rats. This observation may be due to the up-regulated prostaglandins level in this systematic inflammation model (Claveau et al., Microsomal prostaglandin E synthase-1 is a major terminal synthase that is selectively up-regulated during cyclooxygenase-2-dependent prostaglandin E2 production in the rat adjuvant-induced arthritis model. J. Immunol. (2003) 170:4738-4744). It may also help to explain the observed faster polymer extravasation.

Interestingly however, extravasation in the inflamed ankle joints was delayed for a short time (1˜2 h) in the AIA model (FIGS. 3A-3H). The “hot spots” of high MR contrast signal appeared later around the tarsus indicating high local concentrations of P-DOTA-Gd³⁺. These “hot spots” also reveal the locations of possible local damage in and around the joint. The polymer continues to extravasate, diffuse, accumulate in the ankle joints and the greatest concentrations were observed in the 8 hours post injection images (single plane, enlarged MR images, FIG. 4). Because some increased concentrations of polymer were still observed in the joint at 32 hours after injection, it appears that the clearance of the polymer from the joint is relatively slow. By correlating the polymer accumulation, as detected by MRI, with the histology of the same tissues (FIGS. 2A, 2B and 2D), it is evident that the accumulation of the polymer correlates with the degree of inflammation. As observed in the 8 hours MR images, the accumulation of P-DOAT-Gd³⁺ to the knee joints was much less than that observed to the ankle joints (FIG. 4D). This finding agrees very well with the amounts and degree of severity of inflammation observed histologically in the joints (FIG. 2F). In contrast to the observation with AIA rats, no extravasation of P-DOTA-Gd³⁺ to the ankle or knee joints was observed in the healthy control rats.

The data suggests a pharmacokinetic profile with a renal clearance mechanism and a redistribution of the HPMA copolymer (labeled with DOTA-Gd³⁺) from major organs and the blood circulation compartment into the inflammatory arthritic joints. Compared to the normal animal, the result from the MR images of the AIA model clearly demonstrate a very selective polymer targeting and accumulation effect to the arthritic joints with a time frame of about 1 to 2 days after a single bolus injection. Given that most current anti-arthritic drugs do not specifically target the arthritic joints and the damaged tissues, coupled with a low efficacy, the observed targeting and accumulation of the polymeric delivery systems to arthritic joints demonstrate the great effectiveness and numerous potential applications of this invention for the drug delivery and treatment, for example, of rheumatoid arthritis.

Likewise, imaging and evaluation of the inflammatory tissues or organs, such as arthritic joints, with an MRI macromolecular contrast agent, also provides much improved imaging results, as shown in FIGS. 3F & 4C, when compared to the low molecular weight MRI contrast agent, such as OMNISCAN (FIG. 3O). The invention permits a greater time frame for longer and/or more detailed and/or sophisticated imaging process, which cannot be, or are not optimally, performed with the current low molecular weight imaging agents, such as OMNISCAN. More anatomical detail can be revealed with these imaging agents, which may have many applications, such as preclinical evaluation of therapeutic effects of experimental anti-arthritic drugs on an animal model and clinical evaluation of patient response to treatment. Similar benefits may be realized when using the invention with optical, MRI, PET, CT or gamma-scintigraphy imaging agents. When optical, MRI, PET, CT or gamma-scintigraphy imaging agents are conjugated to the polymeric delivery systems described in this invention, they will be able to provide powerful molecular imaging tools for the understanding of inflammatory diseases, such as rheumatoid arthritis.

While the enhanced permeability of the vasculature in the arthritic joints may be comparable to those found in solid tumor, the retention of the polymer in the joint tissue may vary according to the stage of the disease. A swift drug-cleavage mechanism may be applied to ensure effective release of the drug from the macromolecular carrier. A person of ordinary skill in the art will recognize that some pathological features of the arthritic joints may be exploited for this. For example, the release of the drug from the polymer may be facilitated by things such as the very high extracellular enzyme activities (e.g., cathepsins (e.g., cathepsin K), MMPs, etc.) (Okada, Y., Proteinases and Matrix Degradation, In Ruddy et al., (ed.) Kelley's Textbook of Rheumatology, 6^{th} Ed. W.B. Saunders Company, St. Louis, 1997, pp 55-72), low pH, hypoxia or elevated temperature (Treuhaft and McCarty, Synovial fluid pH, lactate, oxygen and carbon dioxide partial pressure in various joint diseases, Arthritis and Rheumatism (1971) 14:475-484; Jayson and Dixon, Intra-articular pressure in the rheumatoid arthritis of knee, I. Pressure changes during passive joint distension, Ann. Rheum. Dis. (1970) 29:261-265). Likewise, measures that would enhance the retention of the extravasated polymers in the joints may also be used according to the invention (e.g., the polymer drug conjugates). Without being bound by theory, internalization by immune cells, such as macrophages, may be considered as the primary cause of polymer retention in the arthritic joints. Incorporation of targeting moieties, which would bind to the negatively charged cartilage (Giraud et al., Application to a cartilage targeting strategy: synthesis and in vivo biodistribution of ¹⁴C-labeled quaternary ammonium-glucosamine conjugates, Bioconjug. Chem. (2000) 11:212-218), the freshly eroded bone surface (Wang et al., Synthesis and evaluation of water-soluble polymeric bone-targeted drug delivery systems, Bioconjug. Chem. (2003) 14:853-859) or the enriched rheumatic factors in the RA joints may also be used to increase the uptake and retention of the polymer in joint tissue. It is also believed that by increasing the molecular weight of the polymeric carrier, a greater retention of the polymer in the RA joint may be accomplished. Anti-arthritic drugs, such as glucocorticoids, can be used in the drug delivery system of the invention.

As will be recognized by a person of ordinary skill in the art, inflammation resolution drugs, anti-inflammatory drugs, anti-arthritic drugs, targeting moieties, and imaging agents, as used herein, include acceptable salts, esters, or salts of such esters. For example, glucocorticoids include pharmaceutically acceptable salts and esters thereof, therefore, when a drug is described, e.g., dexamethasone, pharmaceutically acceptable salts thereof are also described, such as dexamethasone palmitate.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts and acid addition salts are known in the art (see, for example, Berge et al., “Pharmaceutical Salts,” J of Pharma Sci., 1977, 66:1-19; REMINGTON'S PHARMACEUTICAL SCIENCES, 18th Ed. (1990, Mack Publishing Co., Easton, P. A.); and GOODMAN AND GILMAN'S, THE PHARMACOLOGICAL BASIS OF THERAPEUTICS (10th ed. 2001)).

The term “prodrug” indicates a therapeutic agent that is prepared in an inactive form that is converted to an active form (i.e., drug) within the body or cells thereof by the action of endogenous enzymes or other chemicals and/or conditions.

In addition, currently available protein drugs and orally available low molecular weight drug may also benefit from the principles illustrated in the invention. For example, the extravasation of the injected polymer into the RA joints was delayed for 1 to 2 hours. Thus, for the protein or peptide drugs, they must survive this period of time against hepatic and renal clearance. Protein or peptide drugs may be stabilized by methods known in the art, for example, PEGylation of the protein and/or modification of the polymer backbone may provide a beneficial means in solving this problem (Smolen and Steiner, Therapeutic strategies for rheumatoid arthritis. Nature Review Drug Discovery (2003) 2:473-488).

Using modern MR imaging techniques, the specific accumulation of macromolecules was observed in arthritic joints in the rat model of adjuvant-induced arthritis. There was an excellent correlation between the uptake and retention of the MR contrast agent labeled polymer with histopathological features of inflammation and local tissue damage. The methodology used in this study proved that macromolecular imaging agents (polymeric delivery systems conjugated with near IR dyes, MRI, CT PET, gamma-scintigraphy imaging agents) are powerful imaging and evaluation tools for inflammatory diseases, such as rheumatoid arthritis. The use of the macromolecular imaging agents also demonstrates the utility of the delivery system for the purpose of targeting a drug, which is a beneficial improvement over current treatments, for example, for treating rheumatoid arthritis. The invention provides the ability to increase the therapeutic potential and dosing window of the drugs by reducing their side effects. Furthermore, the invention may have a longer half-life in blood circulation when compared to low molecular weight drugs, which may increase the bioavailability of the drug. In addition, the invention may be used to render a hydrophobic drug hydrophilic and, particularly for peptide-based drugs, reduce immunogenecity.

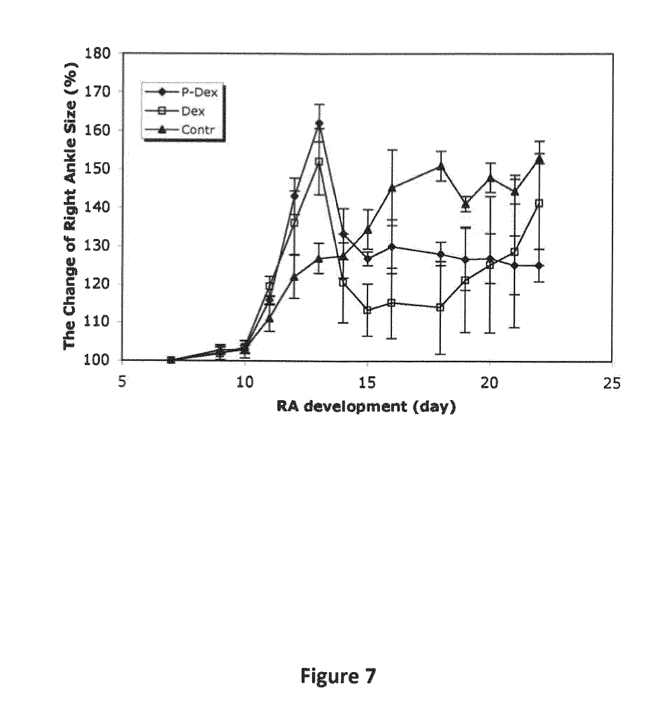

To demonstrate the superior therapeutic effects of the invention, a HPMA copolymer containing targeting moiety with an anti-arthritic drug was synthesized. The anti-arthritic drug, dexamethasone, was linked to the polymer backbone (P-Dex) via a pH sensitive hydrazone bond as illustrated in FIG. 6. The polymer with the hydrazine and dexamethasone attached was then injected into AIA rats (4/group) on day 13 after the induction of arthritis. A single dose of 10 mg (P-Dex)/kg was given. As a control, the same dose of low molecular weight Dexamethasone sodium phosphate (Dex) was divided into 4 equal doses and one dose was given each day to another group of AIA rats (4/group) from day 13-16 after the induction of arthritis. As shown in FIG. 7, both groups of animals showed a dramatic decrease of ankle joint swelling after the injections on day 13. However, with the cessation of the daily injections of the control Dex, the inflammation rapidly got worse while the inflammation in the P-Dex group had a prolonged suppression. These significant advantages of the P-Dex treatment may be attributed to the specific targeting and enhanced retention of the polymeric delivery system to the arthritic joints of the animals.

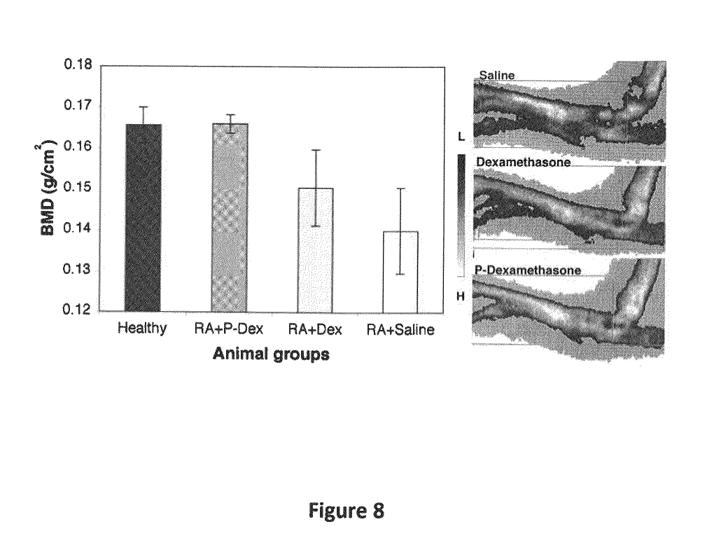

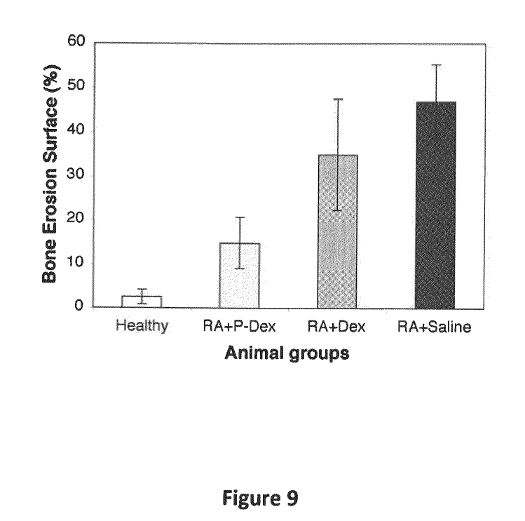

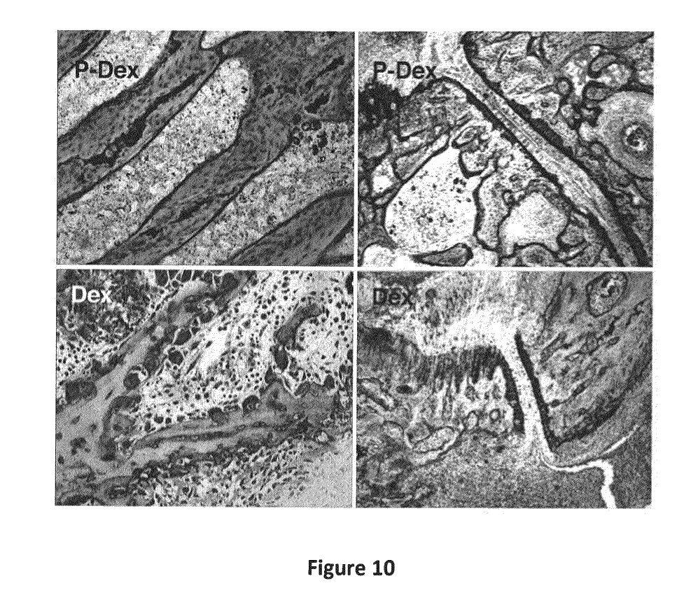

To strengthen the statistics of the observed superior therapeutic effects of the delivery system, a study with larger animal groups (7/group) was performed. One of the significant impacts of rheumatoid arthritis inflammation is the damage to the bone in the joints, which is evident in FIG. 8 of the animals with no treatment (saline). Glucocoticoids, such as dexamethasone (Dex), can slow bone erosion by reducing the inflammation of the joints, as evident in FIG. 8 of animals with Dex treatment. However, such improvement can be greatly strengthened if Dex is conjugated to HPMA copolymer. The inhibition of inflammation is prolonged (FIG. 7) and the bone is well preserved in the P-Dex treated animal group with a BMD similar to the healthy group. A more dynamic factor to consider in the bone metabolism is the extent of bone erosion. The bone erosion surface directly correlates with the recruitment and activity of osteoclasts, which are the cells responsible for bone resorption and the development of bone damage. In FIG. 9, the bone erosion surface data for all the treatment groups is summarized. Again, the P-Dex group showed a lower percentage of erosion surface compare to the Dex group. The histology analysis of the arthritic joints with different treatments also confirmed the superiority of the P-Dex treatment (FIG. 10).

A water-soluble polymer backbone of the invention includes, but is not limited to, a HPMA copolymer and its derivatives, polyethylene glycol (including branched or block copolymers, which may be degradable via peptide sequences, ester or disulfide bonds, etc.), polyglutamic acid, polyaspartic acid, dextran, chitosan, cellulose and its derivatives, starch, gelatin, hyaluronic acid and its derivatives, polymer or copolymers of the following monomers: N-isopropylacrylamide, acrylamide, N,N-dimethylacrylamide, N-vinylpyrrolidone, vinyl acetate (resulting polymer hydrolyzed into polyvinyl alcohol or PVA), 2-methacryloxyethyl glucoside, acrylic acid, methacrylic, vinyl phosphonic acid, styrene sulfonic acid, maleic acid, 2-methacrylloxyethyltrimethylammonium chloride, methacrylamidopropyltrimethyl-ammonium chloride, methacryloylcholine methyl sulfate, N-methylolacrylamide, 2-hydroxy-3-methacryloxypropyltrimethyl ammonium chloride, 2-methacryloxyethyl-trimethylammonium bromide, 2-vinyl-1-methylpyridinium bromide, 4-vinyl-1-methyl-pyridinium bromide, ethyleneimine, (N-acetyl)ethyleneimine, (N-hydroxyethyl)ethyleneimine and/or allylamine. Preferably, the water-soluble polymer is biologically inert, however, optionally the polymer may have therapeutic activity (Rapp et al., Synthesis and in vivo biodisposition of [14C]-quaternary ammonium-melphalan conjugate, a potential cartilage-targeted alkylating drug, Bioconjug Chem. (2003) 14(2):500-6).

The invention may, optionally, include one or more targeting moieties, which may be used to direct the delivery system to a specific tissue, such as bone, cartilage, or certain cell types, etc. Illustrative examples of targeting moieties include, but are not limited to, folic acid, mannose, bisphosphonates, quaternary ammonium groups, peptides (e.g., oligo-Asp or oligo-Glu), aminosalicylic acid, and/or antibodies or fragments or derivatives thereof (e.g., Fab, humanized antibodies, and/or single chain variable fragment (scFv)). A targeting moiety may be linked to the polymer backbone via covalent or physical bonds (linkages). Optionally, the spacers between a targeting moiety and the polymer backbone may be cleaved upon a stimulus including, but not limited to, changes in pH, presence of a specific enzyme activity (for example, cathepsins (e.g., cathepsin K), MMPs, etc.), changes in oxygen levels, etc.

Optionally, the spacers between the therapeutic agent and the polymer backbone may be cleaved upon a stimulus including, but not limited to, changes in pH, presence of a specific enzyme activity (for example, cathepsins (e.g., cathepsin K), MMPs, etc.), changes in oxygen levels, etc.

Optionally, a bio-assay label/imaging agent (or labels) may be attached to the polymer backbone. It may be any label known in the art, including, but not limited to, an optical imaging agent, fluorescent probe, MRI contrast agent, radioisotope, biotin, gold, etc. Their average mol percentage per polymer chain may range from 0% to about 50%.

The bio-assay label, therapeutic agent, and/or targeting moiety may be linked to the water-soluble polymer backbone by way of a spacer. Spacers are known in the art and the person of ordinary skill in the art may select a spacer based on length, reactivity, flexibility and the like. For example, a spacer may be an alkyl or alkyne having from one to 50, preferably one to 15 carbons.

A spacer of the invention may be a peptide sequence (for example, selected from all nature amino acids) having from one to 20, preferably one to 10 residues. In yet another example, a spacer may contain a hydrazone bond which is cleavable under acidic pH. These spacers may be cleaved upon a stimulus including, but not limited to, changes in pH, presence of a specific enzyme activity (for example, cathespins (e.g., cathepsin K), MMPs, etc.), changes in oxygen levels, etc.

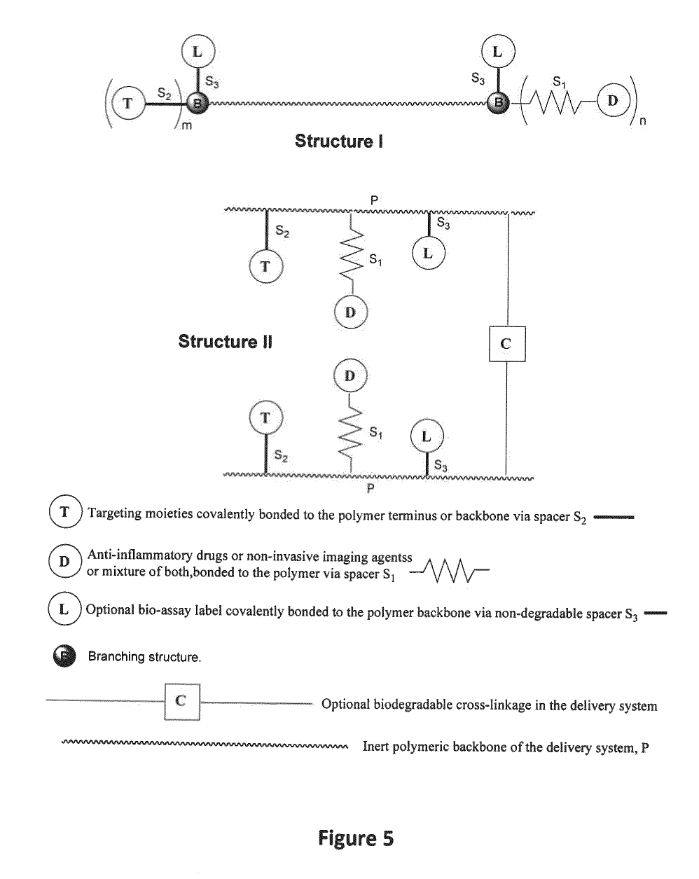

Optionally, the biodegradable cross-linkage shown in FIG. 5 may cross-link, to a certain degree, the linear polymer backbone. The resulting delivery system still retains its water-solubility. The linkage itself is preferably cleavable under physiological conditions.

As will be appreciated by a person of ordinary skill in the art, each class (e.g., therapeutic agent, targeting moiety, bio-assays label and/or imaging agent, spacer) may comprise any number of different compounds or compositions. For example, the therapeutic agent may consist of a mixture of one or more NSAIDs and one or more glucocorticoid, such as a combination of dexamethasone and hydrocortisone. Therefore, the invention provides the advantage that any combination of different therapeutic agents, targeting moieties, bio-assays labels, spacers and/or imaging agents may be incorporated onto the water-soluble polymer backbone. As a result, a drug delivery or imaging system can be created with two or more different therapeutic agents and/or two or more different targeting moieties and/or two or more different bio-assays labels, and/or two or more different spacers (one or more of which may be cleavable, wherein the cleavage stimulus may be different for different spacers) and/or two or more imaging agents. For example, one or more imaging agents may be combined with one or more therapeutic agents, to produce a drug/imaging agent combination, which, for example, may be used to treat and/or monitor the subject. One exemplary embodiment of such a drug/imaging agent is a method of determining the effects of a particular drug or drug combination. For example, the drug/imaging agent may contain a candidate drug wherein the imaging agent allows for enhanced monitoring of the candidate drugs effects. In another exemplary embodiment, the drug/imaging agent may also be used to treat a subject and to monitor the subject's response to the treatment.

An effective amount of a drug is well known in the art and changes due to the age, weight, severity of a subject's condition, the particular compound in use, the strength of the preparation, and the mode of administration. The determination of an effective amount is preferably left to the prudence of a treating physician, but may be determined using methods well known in the art (The Pharmacological Basis of Therapeutics, 10^{th} ed, Gilman et al. eds., McGraw-Hill Press (2001); Remington's Pharmaceutical Science's, 18th ed. Easton: Mack Publishing Co. (1990)). The compositions of the invention may be prepared using methods known in the art, for example, the preparation of a pharmaceutical composition is known in the art (The Pharmacological Basis of Therapeutics, 10^{th} ed, Gilman et al. eds., McGraw-Hill Press (2001); Remington's Pharmaceutical Science's, 18th ed. Easton: Mack Publishing Co. (1990)).

The compositions may be administered by any desirable and appropriate means. For in vivo delivery (i.e., to a subject having arthritis or other inflammatory diseases), it is preferred that the delivery system be biocompatible and preferably biodegradable and non-immunogenic. In addition, it is desirable to deliver a therapeutically effective amount of a compound in a physiologically acceptable carrier. Injection into an individual may occur subcutaneous, intravenously, intramuscularly, intraperitoneal, intraarticular or, for example, directly into a localized area. Alternatively, in vivo delivery may be accomplished by use of a syrup, an elixir, a liquid, a tablet, a pill, a time-release capsule, an aerosol, a transdermal patch, an injection, a drip, an ointment, etc.

### DEFINITIONS

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

The term “isolated” refers to the separation of a compound from other components present during its production. “Isolated” is not meant to exclude artificial or synthetic mixtures with other compounds or materials, or the presence of impurities that do not substantially interfere with the fundamental activity, and that may be present, for example, due to incomplete purification, or the addition of stabilizers.

“Linker”, “linker domain”, and “linkage” refer to a chemical moiety comprising a covalent bond or a chain of atoms that covalently attaches at least two compounds, for example, a targeting moiety to a therapeutic agent. The linker can be linked to any synthetically feasible position of the compounds, but preferably in such a manner as to avoid blocking the compounds desired activity. Linkers are generally known in the art. Exemplary linkers may comprise at least one optionally substituted; saturated or unsaturated; linear, branched or cyclic alkyl group or an optionally substituted aryl group. In a particular embodiment, the linker may contain from 0 (i.e., a bond) to about 500 atoms, about 1 to about 100 atoms, or about 1 to about 50 atoms. The linker may also be a polypeptide (e.g., from about 1 to about 20 amino acids). The linker may be biodegradable under physiological environments or conditions. The linker may also be may be non-degradable and can be a covalent bond or any other chemical structure which cannot be cleaved under physiological environments or conditions.

As used herein, the term “biodegradable” or “biodegradation” is defined as the conversion of materials into less complex intermediates or end products by solubilization hydrolysis under physiological conditions, or by the action of biologically formed entities which can be enzymes or other products of the organism. The term “non-degradable” refers to a chemical structure that cannot be cleaved under physiological condition, even with any external intervention. The term “degradable” refers to the ability of a chemical structure to be cleaved via physical (such as ultrasonication), chemical (such as pH of less than 6 or more than 8) or biological (enzymatic) means.

A “therapeutically effective amount” of a compound or a pharmaceutical composition refers to an amount effective to prevent, inhibit, lessen, or treat the symptoms of a particular disorder or disease.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), bulking substance (e.g., lactose, mannitol), excipient, auxilliary agent or vehicle with which an active agent of the present invention is administered. Pharmaceutically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. The compositions can be incorporated into particulate preparations of polymeric compounds such as polylactic acid, polyglycolic acid, etc., or into liposomes or micelles. Such compositions may influence the physical state, stability, rate of in vivo release, and rate of in vivo clearance of components of a pharmaceutical composition of the present invention. The pharmaceutical composition of the present invention can be prepared, for example, in liquid form, or can be in dried powder form (e.g., lyophilized). Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, 20th Edition, (Lippincott, Williams and Wilkins), 2000; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

The term “alkyl,” as employed herein, includes linear, branched, and cyclic (see cycloalkyl below) chain hydrocarbons containing about 1 to about 20 carbons in the normal chain. An alkyl may be referred to as a hydrocarbyl. Examples of suitable alkyl groups include methyl, ethyl, propyl, isopropyl, butyl, t butyl, isobutyl, pentyl, hexyl, isohexyl, heptyl, 4,4 dimethylpentyl, octyl, 2,2,4 trimethylpentyl, nonyl, decyl, the various branched chain isomers thereof, and the like. Each alkyl group may optionally be substituted (e.g., comprise 1 to about 4 substituents) with at least one substituent which include, for example, halo (such as F, Cl, Br, I), haloalkyl (such as CCl₃ or CF₃), alkyl, alkoxy, hydroxy, aryl, aryloxy, aralkyl, cycloalkyl, alkylamido, alkanoylamino, oxo, acyl, arylcarbonylamino, amino (—NH₂), substituted amino, nitro, cyano, carboxy (—COOH), carbonyl (—C(═O)), epoxy, urea (—NHCONH₂), thiol (—SH), alkylthio, alkyloxycarbonyl (—C(═O)—OR), alkylcarbonyloxy (—OC(═O)—R), carbamoyl (NH₂C(═O)— or NHRC(═O)—), and/or alkylurea (—NHCONHR), wherein R in the aforementioned substituents represents an alkyl radical. The alkyl group may optionally comprise one or more carbon to carbon double bonds (i.e., the alkyl group may be unsaturated). The alkyl may also comprise at least one (e.g., from 1 to about 4) sulfur, oxygen, or nitrogen heteroatoms within the hydrocarbon chain. For example, the alkyl can be —OR, —SR, or —NHR, wherein R is a hydrocarbon chain.

The term “cycloalkyl,” as employed herein, includes saturated and/or unsaturated cyclic hydrocarbon groups containing 1 to 3 rings, that is, monocyclic alkyl, bicyclic alkyl and tricyclic alkyl. Cycloalkyl groups may contain a total of 3 to 20 carbons forming the ring(s), preferably 3 to 10 carbons forming the ring(s), and may optionally be fused to 1 or 2 aromatic rings as described for aryl, below. Unsaturated cycloalkyl groups may contain one or more double bonds and/or triple bonds. Cycloalkyl groups include, without limitation, cyclopropyl, cyclobutyl, cyclopentyl, cyclohexyl, cycloheptyl, cyclooctyl, cyclodecyl and cyclododecyl, cyclopentenyl, cyclohexenyl, cycloheptenyl, cyclooctenyl, cyclohexadienyl, and cycloheptadienyl. Each cycloalkyl group may be optionally substituted (e.g., comprise 1 to about 4 substituents) with substituents (see also above) such as halogen, alkyl, alkoxy, hydroxy, aryl, aryloxy, aralkyl, cycloalkyl, alkylamido, alkanoylamino, oxo, acyl, arylcarbonylamino, amino, substituted amino, nitro, cyano, thiol and/or alkylthio. The cycloalkyl may also comprise at least one (e.g., from 1 to about 4) sulfur, oxygen, or nitrogen heteroatoms within the hydrocarbon chain.

The term “aryl,” as employed herein, refers to monocyclic and bicyclic aromatic groups containing 6 to 10 carbons in the ring portion. Examples of aryl groups include, without limitation, phenyl or naphthyl, such as 1-naphthyl and 2-naphthyl, or indenyl. Aryl groups may optionally include one to three additional rings fused to a cycloalkyl ring or a heterocyclic ring. Aryl groups may be optionally substituted through available carbon atoms with, for example, 1, 2, or 3 groups selected from hydrogen, halo, alkyl, polyhaloalkyl, alkoxy, alkenyl, trifluoromethyl, trifluoromethoxy, alkynyl, aryl, heterocyclo, aralkyl, aryloxy, aryloxyalkyl, aralkoxy, arylthio, arylazo, heterocyclooxy, hydroxy, nitro, cyano, sulfonyl anion, amino, or substituted amino. The aryl group may be a heteroaryl. “Heteroaryl” refers to an optionally substituted, mono-, di-, tri-, or other multicyclic aromatic ring system that includes at least one, and preferably from 1 to about 4, sulfur, oxygen, or nitrogen heteroatom ring members. Heteroaryl groups can have, for example, from about 3 to about 50 carbon atoms (and all combinations and subcombinations of ranges and specific numbers of carbon atoms therein), with from about 4 to about 10 carbons being preferred. Non-limiting examples of heteroaryl groups include pyrryl, furyl, pyridyl, 1,2,4-thiadiazolyl, pyrimidyl, thienyl, isothiazolyl, imidazolyl, tetrazolyl, pyrazinyl, pyrimidyl, quinolyl, isoquinolyl, thiophenyl, benzothienyl, isobenzofuryl, pyrazolyl, indolyl, purinyl, carbazolyl, benzimidazolyl, and isoxazolyl.

The terms “halogen,” “halo,” and “halide” particularly refer to chlorine, bromine, fluorine or iodine.

“Polyethylene glycol,” “PEG,” and “poly(ethylene glycol),” as used herein, refer to compounds of the structure “—(OCH₂CH₂)ₙ—” where (n) ranges from 2 to about 4000. The PEGs of the instant invention may have various terminal or “end capping” groups. The PEGs may be “branched” or “forked”, but are preferably “linear.”

### Polymers and Their Synthesis

In a particular embodiment, the instant invention provides dexamethasone (Dex)-containing N-(2-hydroxypropyl)methacrylamide (HPMA) copolymer conjugates, which are optionally pH-sensitive, with well-defined structure for the improved treatment of rheumatoid arthritis (RA). Indeed, provided are synthesis protocols of new dexamethasone-containing pH-sensitive vinyl monomers, which can be copolymerized directly with other vinyl monomers to obtain polymer-dexamethasone conjugate for the treatment of rheumatoid arthritis and other inflammatory diseases. The pH-sensitive Dex-containing monomer (MA-Gly-Gly-NHN=Dex) can be copolymerized with HPMA using reversible addition-fragmentation transfer (RAFT) polymerization or other living free radical polymerization methods. As described hereinbelow, the structure of the resulting HPMA copolymer-Dex conjugate was analyzed and its therapeutic efficacy was evaluated on adjuvant-induced arthritis (AIA) rats.

The HPMA copolymer-Dex conjugate can be synthesized with controllable molecular weight and polydispersity index (PDI). The Dex content can be controlled by the feed-in ratio of MA-Gly-Gly-NHN=Dex. The HPMA copolymer-Dex conjugate used for in vitro and in vivo evaluation in the examples below has a weight average molecular weight (Mw) of 34 kDa and a PDI of 1.34. The in vitro drug-release studies showed that the Dex release from the conjugate was triggered by low pH. Clinical measurements, endpoint bone mineral density (BMD) test and histology grading from the in vivo evaluation all suggest that newly synthesized HPMA copolymer-Dex conjugate has strong and long-lasting anti-inflammatory and joint protection effects. Thus, the instant invention provides a HPMA copolymer-dexamethasone conjugate with a well-defined structure which has been proven to be an effective anti-arthritis therapy.

In a particular embodiment of the instant invention, the polymers of the instant invention are copolymers comprising a methacrylamide backbone, wherein the methacrylamide units have alkyl or aryl side chains. In a particular embodiment, the amide group of the methacrylamide backbone is omitted. The polymers may comprise at least one therapeutic agent and/or at least one imaging agent. The polymers may further comprise at least one targeting moiety (e.g., linked to the backbone through a linker (e.g., as with the therapeutic agent or imaging agent)). In one embodiment of the instant invention, the polymers of the complexes are block copolymers. Block copolymers are most simply defined as conjugates of at least two different polymer segments (Tirrel, M. In: Interactions of Surfactants with Polymers and Proteins. Goddard E. D. and Ananthapadmanabhan, K. P. (eds.), CRC Press, Boca Raton, Ann Arbor, London, Tokyo, pp. 59-122, 1992). The simplest block copolymer architecture contains two segments joined at their termini to give an A-B type diblock. Consequent conjugation of more than two segments by their termini yields A-B-A type triblock, A-B-A-B-type multiblock, multisegment A-B-C architectures, and the like. More complex architectures such as (AB)ₙ or AₙBₘ starblocks which have more than two polymer segments linked to a single center, are also encompassed by the instant invention. While the methylacrylamide backbone is described throughout, the polymers of the instant invention may also have different backbones such as the backbone generated via click PEG chemistry (see example below).

In another embodiment, the polymer of the instant invention has the general structure:

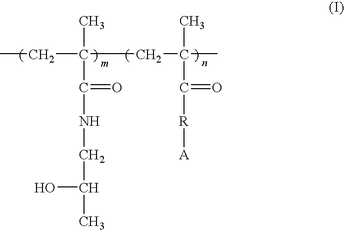

wherein R is a linker; A is an imaging agent or a therapeutic agent; and m and n are independently from about 1 to about 1000, preferably about 10 to about 500. In a particular embodiment, R is an alkyl, aryl, or polypeptide. In a particular embodiment, the R group comprises a pH sensitive linker and/or a cleavable linker. A single copolymer of the instant invention may comprise at least one imaging agent and/or at least one therapeutic agent.

In yet another embodiment, the polymer has the general structure:

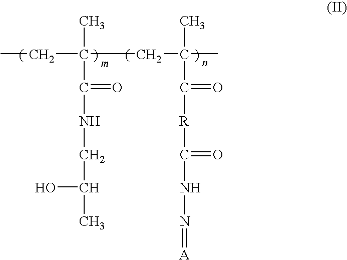

wherein R is a linker; A is a therapeutic agent; and m and n are independently from about 1 to about 1000, preferably about 10 to about 500. In a particular embodiment, R is an alkyl, aryl, or polypeptide. In another embodiment, the R group comprises a pH sensitive linker and/or a cleavable linker.

In still another embodiment, the polymer has the general structure:

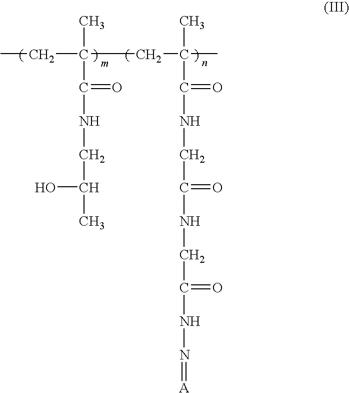

wherein A is a therapeutic agent; and m and n are independently from about 1 to about 1000, preferably about 10 to about 500.

In still another embodiment, the polymer has the structure:

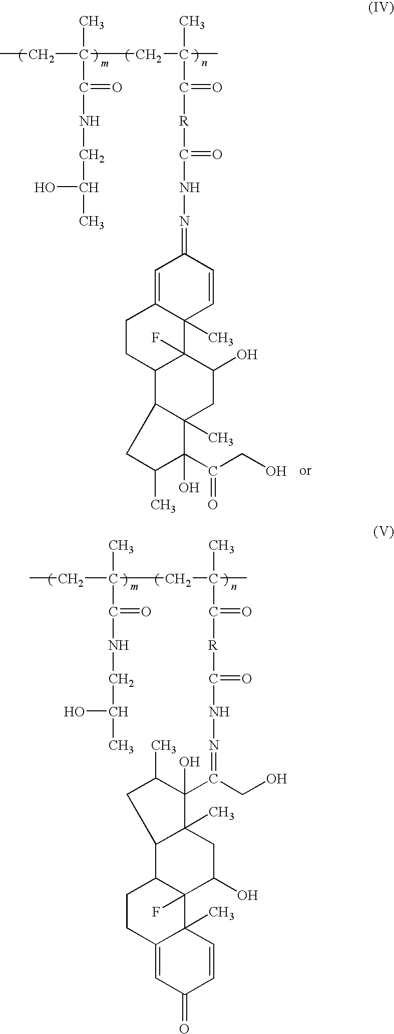

wherein R is a linker and m and n are independently from about 1 to about 1000, preferably about 10 to about 500. In a particular embodiment, R is an alkyl, aryl, or polypeptide. In another embodiment, the R group comprises a pH sensitive linker and/or a cleavable linker. In a particular embodiment, R (optionally including the preceding amine and the following carbonyl) is a polypeptide, particularly a glycine-glycine motif.

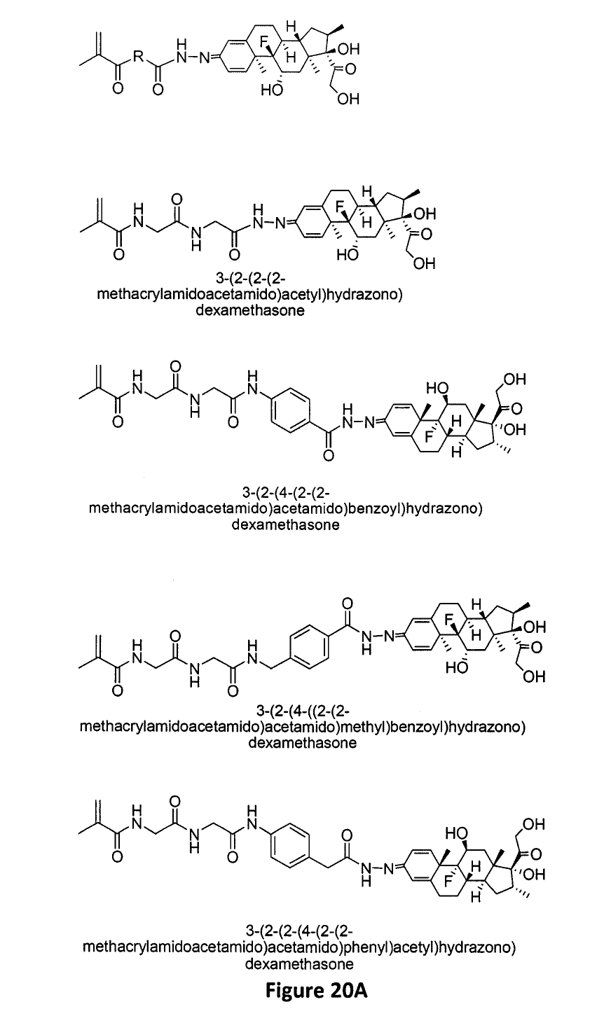

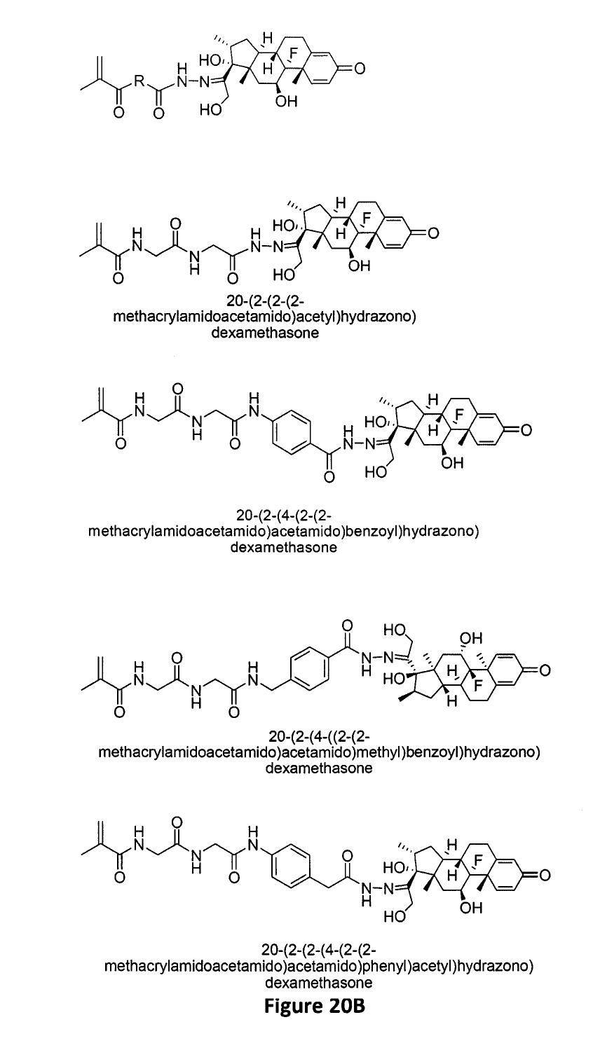

In accordance with another aspect of the instant invention, monomers for the synthesis of the polymers of the instant invention are also provided. In a particular embodiment, the monomers have the general structure:

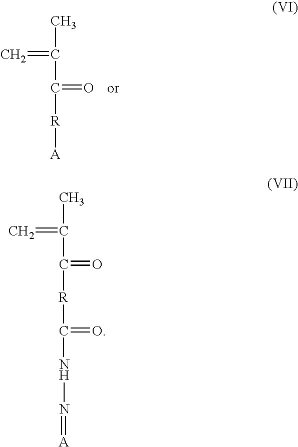

wherein R is a linker and A is an imaging agent or a therapeutic agent. In a particular embodiment, R is an alkyl, aryl, or polypeptide. In yet another embodiment, R comprises an aryl. In a particular embodiment, R (optionally including the following carbonyl) is a polypeptide, particularly a glycine-glycine motif. The R group may comprise a pH sensitive linker and/or a cleavable linker. FIG. 20 provides examples of monomers of the instant invention.

The monomers of the instant invention may be polymerized by any method to form polymers. The polymerization of the monomers and resultant polymers are encompassed by the instant invention. In a particular embodiment, at least one monomer of formula VII is polymerized with at least one monomer of formula VI. Polymerization methods include, without limitation, reversible addition-fragmentation transfer (RAFT) polymerization, atom transfer radical polymerization (ATRP), stable free radical polymerization (SFRP, also called nitroxide mediated polymerization (NMP)), and the like. In a particular embodiment, the monomers are polymerized using reversible addition-fragmentation transfer (RAFT) polymerization.

As stated hereinabove, the polymeric delivery systems of the instant invention can be used for the delivery of at least one therapeutic agent (drug) to the diseased sites for the treatment of inflammatory diseases such as arthritis. In another embodiment, the delivery systems can be used for delivery of at least one imaging agent to the diseased sites for non-invasive imaging and evaluation of the diseased sites of inflammatory diseases such as arthritis. The polymers of the instant invention may each comprise at least one therapeutic agent and/or imaging agent. In another embodiment, multiple polymers are administered (simultaneously or sequentially) each of which comprises a single therapeutic agent and/or imaging agent.

In a particular embodiment, the therapeutic agent attached to the polymer of the instant invention is an anti-inflammatory therapeutic agent. As used herein, an “anti-inflammatory therapeutic agent” refers to compounds for the treatment of an inflammatory disease or the symptoms associated therewith. Anti-inflammatory therapeutic agents include, without limitation, non-steroidal anti-inflammatory drugs (NSAIDs; e.g., aspirin, ibuprofen, naproxen, methyl salicylate, diflunisal, indomethacin, sulindac, diclofenac, ketoprofen, ketorolac, carprofen, fenoprofen, mefenamic acid, piroxicam, meloxicam, methotrexate, celecoxib, valdecoxib, parecoxib, etoricoxib, and nimesulide), corticosteroids (e.g., prednisone, betamethasone, budesonide, cortisone, dexamethasone, hydrocortisone, methylprednisolone, prednisolone, tramcinolone, and fluticasone), rapamycin, rho-kinase inhibitors, viral CC-chemokine inhibitor (vCCIs), glucocorticoids, steroids, beta-agonists, anticholinergic agents, methyl xanthines, sulphasalazine, dapsone, psoralens, proteins, peptides, DMARDs, glucocorticoids, methotrexate, sulfasalazine, chloriquine, gold, gold salt, copper, copper salt, penicillamine, D-penicillamine, cyclosporine, lipoxins, resolving, and protecting. In a particular embodiment, the anti-inflammatory therapeutic agent is selected from the group consisting of proteins, peptides, NSAIDs, DMARDs, glucocorticoids, methotrexate, sulfasalazine, chloriquine, gold, gold salt, copper, copper salt, penicillamine, D-penicillamine, and cyclosporine. In a particular embodiment, the anti-inflammatory therapeutic agent is dexamethasone. Anti-inflammatory therapeutic agents are also provided in The Pharmacological Basis of Therapeutics, 10^{th} ed., Gilman et al., eds., McGraw-Hill Press (2001) and Remington's Pharmaceutical Science's, 18th ed. Easton: Mack Publishing Co. (1990).

In another exemplary embodiment, the invention provides a water-soluble polymeric delivery system for delivery of imaging agents, which are useful for non-invasive imaging, diagnosis, and evaluation of arthritic joints and other inflammatory diseased organs or tissues. The imaging agent containing polymer may also be used to monitor the progress of a treatment. In another exemplary embodiment, the invention provides a method of screening anti-inflammatory therapeutic agents, wherein the anti-inflammatory agent is attached to a water-soluble polymeric delivery system of the invention and administered to a subject, the effect of the therapeutic agent is monitored, for example, using an imaging agent, and an effective therapeutic agent is identified. Alternatively, the anti-inflammatory agent is administered to a patient and an imaging agent attached to a water-soluble polymeric delivery system of the invention is administered so that the effect of the therapeutic agent is monitored. Optionally, an imaging agent may be co-administered for the purpose of monitoring and/or screening the activity of the anti-inflammatory agent. Optionally, a targeting moiety or moieties may be used in the method of screening. The imaging agents may be compounds useful for optical imaging, magnetic resonance imaging (MRI), positron emission tomography (PET), computerized tomography (CT), gamma-scintigraphy imaging, and the like. Such agents are well-known to those of skill in the art. Imaging agents include, without limitation, optical imaging agents (e.g., near IR dyes), MRI enhancing agents (for example, DOTA-Gd³⁺, DTPA-Gd³⁺ (gadolinium complex with diethylenetriamine pentaacetic acid)), PET compounds labeled or complexed with ¹¹C, ¹³N, ¹⁵O, ¹⁸F, ⁶⁴Cu, ⁶⁸Ga, or ⁸²Rb (e.g., ¹⁸F-FDG (fluorodeoxyglucose)), CT compounds (for example, iodine or barium containing compounds, e.g., 2,3,5-triiodobenzoic acid) and gamma-scintigraphy compounds (for example, compounds complexed with ⁹⁹Tc, ¹¹¹In, ¹¹³In, or ¹⁵³Sm).

As stated hereinabove, the polymers of the instant invention are used to treat and/or monitor at least one inflammatory disease/disorder. As used herein, the terms “inflammatory disease” and “inflammatory disorder” refer to a disease or disorder caused by or resulting from or resulting in inflammation. The term “inflammatory disease” may also refer to a dysregulated inflammatory reaction that causes an exaggerated response by macrophages, granulocytes, and/or T-lymphocytes leading to abnormal tissue damage and cell death. An “inflammatory disease” can be either an acute or chronic inflammatory condition and can result from infections or non-infectious causes. Inflammatory diseases and disorders include, without limitation, inflammatory lesions (e.g., those associated with multiple sclerosis, graft or organ transplant rejection, tuberculosis, and the like), atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, eczema, dermatitis, cystic fibrosis, temporomandibular joint syndrome (TMJ), chronic obstructive pulmonary disease, inflamed nerve root, arthrosteitis, rheumatoid arthritis, osteoarthritis (OA), inflammatory arthritis, Sjogren's Syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroditis, Graves' disease, Goodpasture's disease, mixed connective tissue disease, sclerosing cholangitis, inflammatory bowel disease, colitis, Crohn's Disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, emphysema, hayfever, allergies, acute anaphylaxis, rheumatic fever, glomerulonephritis, pyelonephritis, cellulitis, cystitis, chronic cholecystitis, ischemia (ischemic injury and ischemia-reperfusion), hypertension, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, and vulvovaginitis, angitis, chronic bronchitis, osteomylitis, optic neuritis, temporal arteritis, transverse myelitis, necrotizing fascilitis, necrotizing enterocolitis, infection-related disorders such as acute and chronic bacterial and viral infections and sepsis, and neoplasia (including leukocyte recruitment in cancer and angiogenesis). In a particular embodiment, the inflammatory disease is selected from the group consisting of rheumatoid arthritis, osteoarthritis (OA), temporomandibular joint syndrome (TMJ), inflamed nerve root, Crohn's disease, chronic obstructive pulmonary disease, psoriasis diseases, asthma, colitis, multiple sclerosis, lupus, erythematosus, and atherosclerosis. In one embodiment, the inflammatory disease is rheumatoid arthritis.

The linkages (linker domains) of the instant polymers may be non-degradable or degradable under physiological conditions. The linkers may be cleavable by a protease such as cathepsins and MMPS. The linkers may also be pH sensitive (e.g., cleaved under acidic conditions (e.g., pH<6, preferably <5.5). In a particular embodiment, the pH sensitive linker comprises a hydrazone bond, acetal bond, cis-aconityl spacer, phosphamide bond, silyl ether bond, and the like.

The instant invention also encompasses compositions comprising at least copolymer of the instant invention and at least one pharmaceutically acceptable carrier. The composition may further comprise at least one other anti-inflammatory therapeutic agent. Such composition may be administered, in a therapeutically effective amount, to a patient in need thereof for the treatment and/or imaging of an inflammatory disease or disorder. In a particular embodiment, at least one other anti-inflammatory agent is administered separately from the above composition (e.g., sequentially or concurrently).

The compositions of the present invention can be administered by any suitable route, for example, by injection (e.g., for local (direct) or systemic administration (intravenous)), oral, pulmonary, topical, nasal or other modes of administration. The composition may be administered by any suitable means, including parenteral, intramuscular, intravenous, intraarterial, intraperitoneal, subcutaneous, topical, inhalatory, transdermal, intraocular, intrapulmonary, intraareterial, intrarectal, intramuscular, and intranasal administration. In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., Tween 80, Polysorbate 80), anti oxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington's Pharmaceutical Sciences, 18th Ed. (1990, Mack Publishing Co., Easton, Pa. 18042) pages 1435 1712 which are herein incorporated by reference. The pharmaceutical composition of the present invention can be prepared, for example, in liquid form, or can be in dried powder form (e.g., lyophilized).

In a particular embodiment, the composition may be administered topically. The composition for topical administration may be formulated, for example, as a cream, lotion, foam, or ointment. As another example, inflammations of the joints or tendons (e.g., arthritis, tendonitis) may be treated by injecting the composition directly into the affected location. Such injections may be administered at intervals until inflammation has subsided. As yet another example, inflammatory conditions of the airways or lungs (e.g., asthma) may be treated by inhalation therapy with an aerosol formulation of the composition. As still another example, inflammations or autoimmune diseases of the gastrointestinal tract (e.g., irritable bowel syndrome, Crohn's Disease) may be treated orally with the composition formulated as a pill, powder, capsule, tablet, or liquid to coat the lumenal surface of the gastrointestinal tract. As another example, the composition may be administered systemically (e.g., intravenously) for treatment of inflammatory disorders that are, at least in part, systemic in nature (e.g., systemic lupus, multiple sclerosis, rheumatoid arthritis, dermatomyositis and scleroderma) or that do not lend themselves well to localized drug delivery.

In yet another embodiment, the pharmaceutical compositions of the present invention can be delivered in a controlled release system, such as using an intravenous infusion, an implantable osmotic pump, a transdermal patch, liposomes, or other modes of administration. In a particular embodiment, a pump may be used (see Langer, supra; Sefton, CRC Crit. Ref. Biomed. Eng. (1987) 14:201; Buchwald et al., Surgery (1980) 88:507; Saudek et al., N. Engl. J. Med. (1989) 321:574). In another embodiment, polymeric materials may be employed (see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. (1983) 23:61; see also Levy et al., Science (1985) 228:190; During et al., Ann. Neurol. (1989) 25:351; Howard et al., J. Neurosurg. (1989) 71:105). In yet another embodiment, a controlled release system can be placed in proximity of the target tissues of the animal, thus requiring only a fraction of the systemic dose (see, e.g., Goodson, in Medical Applications of Controlled Release, supra, (1984) vol. 2, pp. 115 138). In particular, a controlled release device can be introduced into an animal in proximity to the site of inappropriate inflammation. Other controlled release systems are discussed in the review by Langer (Science (1990) 249:1527 1533).

The composition of the instant invention may be administered for immediate relief of acute symptoms or may be administered regularly over a time course to treat and/or image the inflammatory disorder. The dosage ranges for the administration of the composition of the invention are those large enough to produce the desired effect (e.g., curing, relieving, and/or preventing the inflammatory disorder, the symptom of it, or the predisposition towards it). The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications.

The following examples are provided to illustrate various embodiments of the present invention. They are not intended to limit the invention in any way.

### Example 1

### Introduction

Rheumatoid arthritis (RA) is a chronic inflammatory disease of unknown etiology and complex multifactorial pathogenesis, affecting approximately 0.8 percent of adults worldwide. RA is characterized by destructive inflammation of joints, with the eventual deterioration of the articular bone and cartilage (Lawrence et al. (1998) Arthritis Rheum., 41:778-799; Firestein, G. S., Etiology and pathogenesis of rheumatoid arthritis. In Kelley's Textbook of Rheumatology 7th edition. Edited by: Harris et al., Philadelphia: Elsevier Saunders. 996-1042 (2005)). Improved understanding of pathophysiology of RA has led to several effective therapeutic strategies for the treatment of RA, such as nonsteroidal anti-inflammatory drugs (NSAIDs), glucocorticoids (GCs), and disease-modifying anti-rheumatic drugs (DMARDs) (Smolen et al. (2003) Nat Rev Drug Discov., 2: 473-488; O'Dell, J. R. (2004) N. Engl. J. Med. 350:2591-2602). These drugs are relatively safe and effective in the short-to-intermediate-term treatment of RA. However, long-term use may result in various, sometimes severe, side effects (FitzGerald, G. A. (2003) Nat Rev Drug Discov., 2:879-890; Baylink, D. J. (1983) N Engl J. Med., 309:306-308; Borchers et al. (2004) Semin Arthritis Rheum., 34:465-483). While the management of RA continues to evolve with many new drugs (e.g. gene therapy) under investigation (Smolen et al. (2003) Nat Rev Drug Discov., 2: 473-488; O'Dell, J. R. (2004) N. Engl. J. Med. 350:2591-2602; Olsen et al. (2004) N. Engl. J. Med., 350:2167-2179; Traister et al. (2008) Mod. Rheumatol., 18:2-14), most of them do not have arthrotropicity. Generally, this lack of tissue specificity combined with the ubiquitous distribution of the molecular targets may explain the significant systemic and extra-articular adverse events often associated with antirheumatic drugs (Capell, H. (2002) J. Rheumatol. Suppl., 66:38-43; Garrood et al. (2006) Arthritis Rheum., 54:1198-1208).

The discovery of novel joint-specific molecular targets may address this problem head-on. From a practical angle, however, drug delivery offers a simple solution by incorporating the arthrotropism to available antirheumatic drugs (Boerman et al. (1997) Ann. Rheum. Dis., 56:369-373; Wang et al. (2004) Pharm. Res., 21:1741-1749). These newly developed strategies capitalize on two unique pathophysiological conditions of RA joints, the enhanced vascular permeability to macromolecules, and the relatively acidic local environment. RA is characterized by synovial proliferation with chronic inflammatory cell infiltration and neovascularization (Koch, et al. (2003) Ann. Rheum. Dis., 62 Suppl 2:ii60-67). These pathologic neoangiogenic vessels are similar to that in tumors and tend to exhibit disordered architecture and have enhanced permeability to macromolecules compared with normal vessels (Matsumura et al. (1986) Cancer Res., 46:6387-6392; Levick, J. R. (1981) Arthritis Rheum., 24:1550-60). The local inflammatory reaction in and around RA joint tissues also promotes an acidic environment known as acidosis. This is partially due to the low levels of oxygen in the synovial fluid, which appears to induce a shift towards anaerobic glycolysis and lactate formation (Levick, J. R. (1990) J. Rheumatol., 17:579-582; Falchuk et al. (1970) Am. J. Med., 49:223-231). The pH values of synovial fluid had been reported as low as 6.0 (Goldie et al. (1969) Acta Orthop. Scand., 40:634-641). Much lower pH values (4.4-5.6) in the synovial tissue have also been reported (Andersson et al. (1999) J. Rheumatol., 26:2018-2024; Konttinen et al. (2002) Arthritis Rheum., 46:953-960; Konttinen et al. (2001) J. Bone Miner. Res., 16:1780-1786).

The leaky vasculature of RA joint has been successfully exploited in targeted glucocorticoid therapy. Long-circulating liposomes loaded with GC could remain in the circulation with long half-life and extravasate selectively into the inflamed joints with high concentrations. These formulations may increase the therapeutic index of GCs and enable their use as both short-term treatment and prolonged therapy for RA (Schmidt et al. (2003) Brain 126:1895-1904; Metselaar et al. (2003) Arthritis Rheum. 48:2059-66; Metselaar et al. (2004) Ann. Rheum. Dis., 63:348-353; Avnir et al. (2008) Arthritis Rheum., 58:119-129).

Similar to the liposome approach, it has been determined that synthetic water-soluble polymers, such as N-(2-hydroxypropyl)methacrylamide (HPMA) copolymers have strong arthrotopicity when administered to adjuvant-induced arthritis rats (AIA) (Wang et al. (2004) Pharm. Res., 21:1741-1749). Based upon this finding, a HPMA copolymer-dexamethasone conjugate (P-Dex, copolymer of MA-Gly-Gly-NHN=Dex and HPMA) with a pH-sensitive drug activation mechanism has been developed that would further enhance the RA joint specificity of the delivery system (U.S. Pat. No. 4,034,040). Results from initial in vivo evaluation suggested that the conjugate offers superior and longer-lasting anti-inflammatory effects when compared with free dexamethasome (Dex). Greater bone and cartilage preservation is also observed with the P-Dex treatment. However, the inconsistency of drug-loading from batch to batch is a challenge that may hamper its translation into clinical application. This problem is largely due to the synthetic strategy that has been employed. As the modification of the HPMA copolymer precursor proceeds, unreacted pendent functionalities will be left with each polymer analogous reaction step. Regardless, the final Dex loading is hard to control.

To address this issue, a new pH-sensitive Dex-containing monomer has been designed, synthesized and reported hereinbelow. Direct copolymerization of this monomer with HPMA allows easy control of Dex loading in the conjugate. No unreacted pendent functionalities will exist in the drug conjugate. Reversible addition-fragmentation transfer (RAFT) polymerization was used to copolymerize the novel Dex-containing monomer with HPMA. This method provides better control of the polydispersity of the polymer drug conjugate. The therapeutic efficacy of this novel HPMA copolymer-Dex conjugate was evaluated using AIA rats.

### Materials and Methods

### Materials

S,S′-Bis(α,α′-dimethyl-α″-acetic acid)-trithiocarbonate (Lai et al. (2002) Macromolecules 35:6754-6756), N,N-dioctadecyl-N′,N′-bis(2-hydroxyethyl)-1,3-propanediamine (LA) (U.S. Pat. No. 4,034,040), HPMA (Kopecek et al. (1973) Eur. Polym. J. 9:7-14), N-methacryloylaminopropyl fluorescein thiourea (MA-FITC) (Omelyanenko et al. (1998) J. Control. Release 53:25-37) and N-methacryloylglycylglycine (MA-Gly-Gly-OH) (Rejmanová et al. (1977) Makromol. Chem., 178:2159-2168) were prepared as described previously. Sephadex LH-20 resin was obtained from GE Healthcare (Piscataway, N.J., USA). Mycobacterium tuberculosis H37Ra (heat-killed, desiccated) was obtained from VWR International (West Chester, Pa., USA). Paraffin oil (low viscosity, Bayol F) was obtained from Crescent Chemical Company, Inc. (Islandia, N.Y., USA). Dexamethasone (Dex) was purchased from Hawkins, Inc. (Minneapolis, Minn., USA). All solvents and other reagents if not specified were purchased from Fisher Scientific (Pittsburgh, Pa., USA) and used without further purification.

### Instruments

¹H and ¹³C NMR spectra were recorded on a 500 MHz NMR spectrometer (Varian, Palo Alto, Calif., USA). The weight average molecular weight (M_{w}) and number average molecular weight (Mₙ) of copolymers were determined by size exclusion chromatography (SEC) using the ÄKTA FPLC system (GE HealthCare) equipped with UV and RI (KNAUER, Berlin, Germany) detectors. SEC measurements were performed on Superdex 200 column (HR 10/30) with phosphate-buffered saline (pH=7.3) as the eluent. HPMA homopolymer (PHPMA) samples with narrow polydispersity were used as calibration standards. HPLC analyses were performed on an Agilent 1100 HPLC system (Agilent Technologies, Inc., Santa Clara, Calif., USA) with a reverse phase C₁₈ column (Agilent, 4.6×250 mm, 5 μm). Bone mineral density (BMD) was measured with a PDEXA® (peripheral dual energy x-ray absorptiometry) Sabre™ X-ray bone densitometer (Norland Medical System, Inc., Fort Atkinson, Wis., USA)

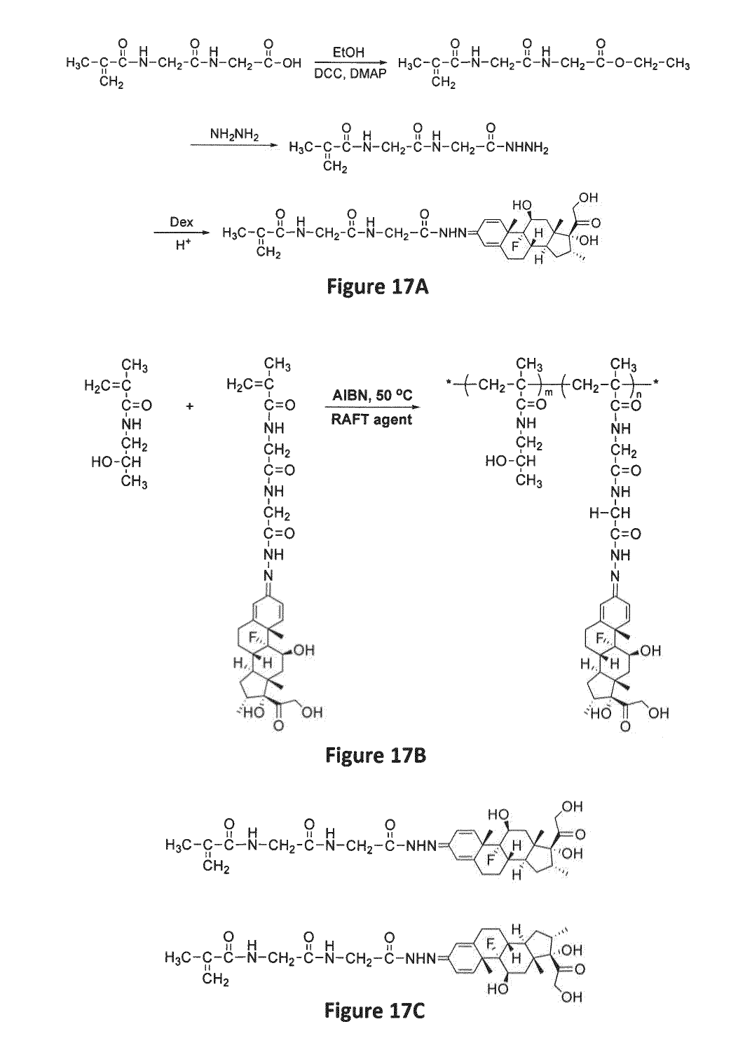

Synthesis of N-methacryloylglycylglycyl hydrazide (MA-Gly-Gly-NHNH₂, Scheme 1, FIG. 17A)

N-Methacryloylglycylglycine (MA-Gly-Gly-OH, 0.8 g, 4 mmol) was suspended in ethanol (15 mL) at 0° C. Small amount of inhibitor (tert-octyl pyrocatechine) was added into the solution to prevent polymerization. N,N′-Dicyclohexylcarbodiimide (DCC, 0.9 g, 4.4 mmol) in ethanol (5 mL) was added into the reaction solution.

The reaction mixture was stirred for 2 hours at 0° C. and 2 hours at room temperature and then filtered to remove dicyclohexylurea (DCU) at 0° C. Hydrazine hydrate (0.4 mL) in ethanol (5 mL) was then added into the filtrate and the solution was stirred for 4 hours at room temperature. When crystalline material started to precipitate, hexane (25 mL) was added and the final product was filtered and washed with ethanol-hexane (1:1, v/v). Yield: 60%.

¹H NMR (d₆-DMSO) δ (ppm): 8.94 (s, 1H), 8.15 (t, J=5.9 Hz, 1H), 8.07 (t, J=5.9 Hz, 1H), 5.74 (s, 1H), 5.38, (s, 1H), 4.20 (s, 2H), 3.76 (d, J=5.9 Hz, 2H), 3.67 (d, J=5.9 Hz, 2H), 1.88 (s, 3H); ¹³C NMR (d₆-DMSO) δ (ppm): 169.5, 168.3, 167.9, 139.6, 120.0, 42.7, 41.1, 18.8.

Synthesis of pH-sensitive, Dex-containing monomer (MA-Gly-Gly-NHN=Dex, Scheme 1)

N-Methacryloylglycylglycyl hydrazide (200 mg, 1 mmol) and Dex (390 mg, 1 mmol) were dissolved in methanol (9 mL). Acetic acid (0.5 mL) was added to the reaction solution as a catalyst. The solution was purged with Argon and stirred for 3 days at room temperature in a sealed ampule. After evaporation of the reaction solvent, the product was purified by flash column chromatography (chloroform/ethanol=8:1, v/v). Yield: 30%.

¹H NMR (d₆-DMSO) δ (ppm): 10.84 (s, 0.5H), 10.49 (s, 0.5H), 8.19 (t, J=6.3 Hz, 1H), 8.10 (t, J=6.3 Hz, 0.5H), 7.88 (t, J=6.3 Hz, 0.5H), 6.78 (s, 0.5H), 6.68 (s, 0.5H), 6.45 (dd, J₁=15.6 Hz, J₂=10.2 Hz, 1H), 6.24 (dd, J₁=24.9 Hz, J₂=10.2 Hz, 1H), 5.73 (s, 1H), 5.37 (s, 1H), 5.12 (br, 1H), 4.92 (s, 1H), 4.66 (br, 1H), 4.48 (dd, J₁=19.0 Hz, J₂=5.4 Hz, 1H), 4.09 (m, 3H), 3.86 (m, 1H), 3.77 (m, 2H), 2.93 (m, 1H), 2.61 (m, 1H), 2.19 (m, 4H), 1.87 (s, 3H), 1.72 (m, 1H), 1.60 (q, 1H, 10.6 Hz), 1.41 (s, 3H), 1.20 (m, 3H), 0.84 (s, 3H), 0.77 (d, 3H, J=6.8 Hz); ¹³C NMR (d₆-DMSO) δ (ppm): 211.4, 170.6, 169.5 (d, syn and anti conformation), 167.8 (d, syn and anti conformation), 165.6, 157.2 (d, syn and anti conformation), 143.1, 139.7 (d, syn and anti conformation), 139.0, 126.5, 119.9 (d, syn and anti conformation), 100.8 (d, J_{CF}=173 Hz), 90.3, 70.1 (d, J_{CF}=37 Hz), 66.5, 47.6, 47.3 (d, J_{CF}=22 Hz), 43.6, 42.5 (d, syn and anti conformation), 40.6 (d, syn and anti conformation), 36.0, 35.1, 34.0 (d, J_{CF}=20 Hz), 32.2, 31.0, 27.5, 24.4 (d, J_{CF}=6 Hz), 18.7, 16.8, 15.5.

Synthesis of HPMA copolymer-Dex conjugate (P-Dex) via RAFT copolymerization (Scheme 2, FIG. 17B)

HPMA (444 mg, 3.1 mmol) and MA-Gly-Gly-NHN=Dex (128 mg, 0.22 mmol, N-(2-(2-(2-((8S,9R,10S,11S,13S,14S,16R,17R)-9-fluoro-11,17-dihydroxy-17-(2-hydroxyacetyl)-10,13,16-trimethyl-7,8,11,12,13,15,16,17-octahydro-6H-cyclopenta[a]phenanthren-3(9H,10H,14H)-ylidene)hydrazinyl)-2-oxoethylamino)-2-oxoethyl)methacrylamide) were dissolved in methanol/DMF (6:1, v/v), with 2,2′-azobisisobutyronitrile (AIBN, 2.7 mg, 0.016 mmol) as initiator and S,S′-bis(α, α′-dimethyl-α″-acetic acid)-trithiocarbonate as RAFT agent. Trace amount of MA-FITC was also added into the copolymerization solution to afford the final product a fluorescent tag for easy detection in purification. The solution was purged with Argon and polymerized at 50° C. for 2 days. The resulting polymer was first purified on a LH-20 column to remove the unreacted low molecular weight compounds, and then dialyzed. The molecular weight cutoff of the dialysis tubing is 25 kDa of globular protein. The polymer solution was lyophilized to obtain the final P-Dex. Yield: 250 mg.

To quantify Dex loading in P-Dex, it was hydrolyzed in 0.1 N HCl (1 mg/mL) overnight. The resulting solution was neutralized and analyzed with HPLC. Mobile phase, acetonitrile/water=2/3; Detection, UV 240 nm; Flow rate, 1 mL/min; Injection volume: 10 μL. The analyses were performed in triplicate. The mean value and standard deviation were obtained with Microsoft Excel.

In Vitro Dex Release from P-Dex

P-Dex (2 mg/mL) was dissolved in acetate buffer (0.01 M with 0.15 M NaCl, pH 5.0) or phosphate buffer (0.01 M with 0.15 M NaCl, pH 7.4) and incubated at 37° C. At selected time intervals, the P-Dex solution (0.3 mL) was withdrawn and neutralized for HPLC analysis. The analysis of each sample was performed in triplicate. The mean value and standard deviation were obtained with Microsoft Excel.

Treatment of AIA Rats with P-Dex

Male Lewis rats (175 to 200 g) were obtained from Charles River Laboratories, Inc. (Wilmington, Mass., USA) and allowed to acclimate for at least 1 week. To induce arthritis, Mycobacterium tuberculosis H37Ra (1 mg) and LA (5 mg) were mixed in paraffin oil (100 μL), sonicated and injected subcutaneously into the base of the tail (Chang et al. (1980) Arthritis Rheum., 23:62-71). The progression of the joint inflammation was monitored daily. Special care was given to the rats as the inflammation developed to ensure access to water and food. All animal experiments were performed according to a protocol approved by the University of Nebraska Medical Center Institutional Animal Care and Use Committee and adhered to Principles of Laboratory Animal Care (National Institutes of Health publication 85-23, revised in 1985).

Rats with established arthritis were selected and randomly assigned into three groups, P-Dex, free Dex, and saline, with 6-7 AIA rats per group. Six healthy, untreated rats were also included as a control group. On the 14^{th} day post arthritis induction, P-Dex (100 mg/kg, [Dex]_{P-Dex}=100 mg/g of P-Dex) was given intravenously to one group of AIA rats. An equivalent dose of free Dex (10 mg/kg of free Dex in the form of water-soluble dexamethasone sodium phosphate) was divided into four aliquots (2.5 mg/kg of free Dex) and was administered (i.p.) to the second group of AIA rats on days 14, 15, 16, and 17. Saline was given similarly to the third group of AIA rats. The clinical measurements of ankle joints were performed daily. On day 24, all animals were euthanized. The hind limbs were dissected at the knee joint. The BMDs (right leg) of the region from distal tibia to the phalanges of the foot were measured by peripheral dual energy x-ray absorptiometry (pDEXA).

### Clinical Measurements

The clinical parameters measured during the treatment included articular index (AI) score and ankle diameter. AI scores were taken for each hind paw by the same observer from day 8 to 24 and the sum of the score from each animal was recorded. The AI scoring for arthritis was performed using a 0-4 scale, where 0=no signs of swelling or erythema, 1=slight swelling and/or erythema, 2=low-to-moderate edema and signs involving the tarsals (proximal part of the hind paw), 3=pronounced edema with limited use of the joint and signs extending to the metatarsals, 4=excessive edema with joint rigidity and severe signs involving the entire hind paw. Clipper measurements of ankle diameter (medial to lateral) were taken every day from day 8 to 24 using a digital caliper (World Prescision Instruments, Inc. Saraspta, Fla., USA).

### Histological Analysis

The hind limbs were isolated and fixed with buffered formalin (10%) for 3 days and decalcified with 12.5% (v/v) HCl for at least 2 more days. The decalcification solution was replaced with fresh solution every 24 hours. When decalcification was complete, the ankle joint was transected along the longitudinal plane to give approximately equal halves. Each half joint was then embedded in paraffin. Sections (5 μm thickness) from each ankle joint were cut approximately 200 μm apart and stained using a standard hematoxylin and eosin (H & E) staining method. The joints were histologically scored according to a grading system modified from the literature (van Dijke et al. (1999) Magn. Reson. Imaging. 17:237-245). The histological changes of joints were graded on the following parameters: synovial cell lining hyperplasia (0-2); villous hyperplasia (0-3); mononuclear cell infiltration (0-3); polymorphnuclear leukocytes infiltration in periarticular soft tissue (0-3); cellular infiltration and bone erosion at the distal tibia (0-2) and cellular infiltration of cartilage (0-1) (Table 1). The histology score was then recorded for each ankle joint and summarized for each animal by two independent examiners, who were blinded to the treatment the animal received.

TABLE 1
Histological grading system, modified from the method from
van Dijke et al. (1999) Magn. Reson. Imaging., 17: 237-245.

| Parameters | Grade | Features |
|---|---|---|
| Synovial cell lining | 0 | 1 to 3 layers of synoviocytes |
| hyperplasia | 1 | 4 to 6 layers of synoviocytes |
| | 2 | 7 or more layers of synoviocytes |
| Villous hyperplasia | 0 | Not present |
| | 1 | Few, scattered, and short |
| | 2 | Moderate and finger-like (form pannus) |
| | 3 | More, clustered, and diffuse |
| Cellular infiltration | 0 | Normal |
| of mononuclear cells | 1 | Mild |
| | 2 | Moderate |
| | 3 | Severe |
| Polymorphonuclear | 0 | Normal |
| leukocyte infiltration | 1 | Mild |
| into periarticular | 2 | Moderate |
| soft tissue | 3 | Severe |
| Cellular infiltration | 0 | Not present |
| and bone erosion at | 1 | Mild |
| the distal tibia | 2 | Moderate |
| Cellular infiltration | 0 | Not present |
| of cartilage | 1 | Present |

### Statistical Methods

One-way analysis of variance (ANOVA) was performed followed by a post hoc test (Tukey-Kramer) for multiple comparisons using Instant Biostatistics (Version 3.0, GraphPad Software, La Jolla, Calif., USA). A value of P<0.05 was considered statistically significant.

### Results

### Syntheses of MA-Gly-Gly-NHN=Dex and P-Dex

As the first step in synthesis of MA-Gly-Gly-NHN=Dex, MA-Gly-Gly-NHNH₂ must be obtained. MA-Gly-Gly-OH was reacted with ethanol to form an ethyl ester. It was then hydrazinolyzed to obtain MA-Gly-Gly-NHNH₂ with about 60% of yield. This route was proved to be very simple with easy purification workup. To obtain the final hydrazone-containing Dex monomer, Dex was initially reacted with MA-Gly-Gly-NHNH₂ in DMF using acetic acid or HCl as a catalyst. The reaction was not successful. Switching solvent to methanol greatly improved the efficiency of reaction. After flash column chromatography, the final product was obtained with 30% of yield. The structures of MA-Gly-Gly-NHN=Dex (syn/anti diastereomers, Scheme 3, FIG. 17C) were confirmed with ¹H and ¹³C NMR spectra. No attempt was made to separate the diastereomers. Using AIBN as the initiator and S,S′-bis(α, α′-dimethyl-α″-acetic acid)-trithiocarbonate as the RAFT agent, MA-Gly-Gly-NHN=Dex was copolymerized with HPMA. After purification with LH-20 column and dialysis, the FPLC analysis results showed that the weight average molecular weight (M_{w}) of P-Dex is 34 kDa with a polydispersity index (PDI) of 1.34, which is narrower than a regular free radical polymerization of HPMA (PDI=1.5-1.6). The Dex content in the P-Dex was determined as 100 mg/g of P-Dex. Conversion of MA-Gly-Gly-NHN=Dex in the copolymerization is about 50%. When the feed-in ratio of Dex monomer changed, the convention ratio was kept at about 50%. The HPLC analysis showed that 99.2% of Dex content was covalently conjugated to the polymer. The 0.8% free Dex detected in the purified P-Dex may come from the dialysis step. This conjugate was used for in vitro Dex release study and in vivo treatment study of the AIA rats.

In Vitro Dex Release from P-Dex

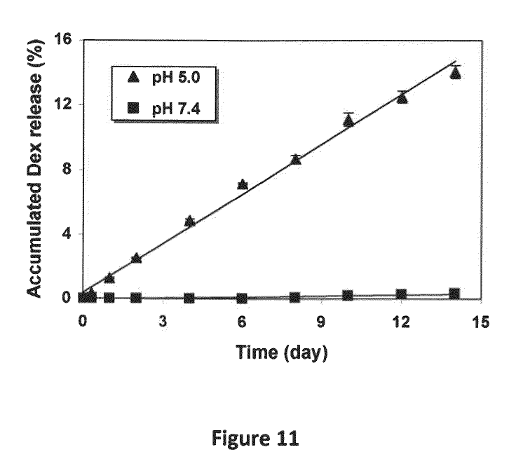

The in vitro Dex release was studied by incubating P-Dex in pH 5.0 and pH 7.4 at 37° C. (FIG. 11). Under neutral condition, there is almost no Dex release from P-Dex; while at pH 5.0, Dex was gradually released from the conjugate. During the course of the experiment (14 days), P-Dex demonstrated a zero-order release at pH 5.0. The release rate of Dex from P-Dex is about 1% of the loaded drug per day.

Clinical Evaluations of AIA Rats Treated with P-Dex, Free Dex and Saline

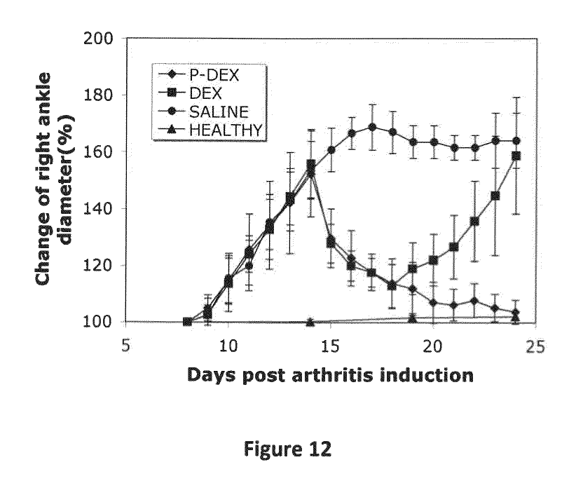

The therapeutic effect of P-Dex was evaluated on AIA rats in comparison with free Dex, saline and healthy controls. On day 8 to 9 post arthritis induction, the disease started with the onset of mild swelling and increased ankle diameter (FIGS. 12 & 13). The ankle joint inflammation continue to become worse, and by day 13 to 14, the swelling started to plateau. All treatments were initiated on day 14. The response to treatments by both free Dex and P-Dex were immediate and very significant. As shown in FIGS. 12 & 13, ankle size and AI scores for these two groups were greatly reduced on day 15. The animals were more mobile and active. Upon cessation of the treatment on day 18, inflammatory flare was observed, with a rapid decrease in mobility in the free Dex group. By the end of the study on day 24, the animals that had been treated with free Dex were indistinguishable from those in the saline group in terms of ankle diameter and AI scores.

Contrary to the free Dex treatment, single P-Dex treatment strongly suppressed the ankle joint inflammation during the entire course of the treatment. It was maintained at the end of the study (euthanasia on day 24).

### BMD Assessment

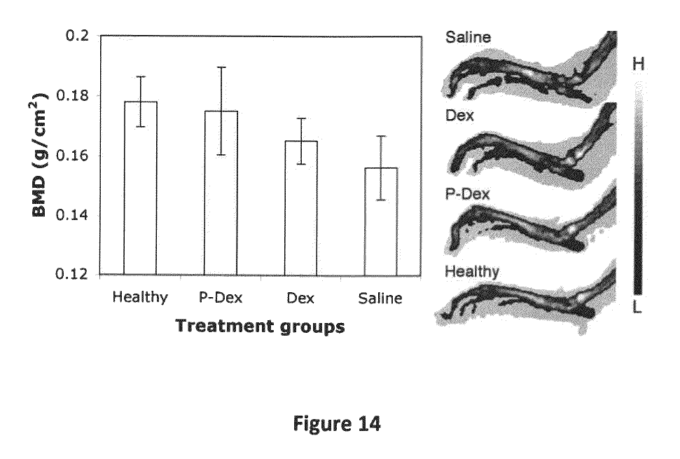

The measurement of BMD from distal tibia to the phalanges of the foot of all animals was performed at the end of the study (FIG. 14). The saline group was found to have the lowest mean BMD values of 0.156, followed by free Dex group (BMD=0.165) and P-Dex group (BMD=0.175). The healthy control group showed the highest mean BMD value of 0.178. From the pDEXA images, it was determined that the major bone destruction of arthritic joints happened at distal tibia, with minor BMD reduction at carpal and metacarpal bones.

### Histological Evaluation of Ankle Joints

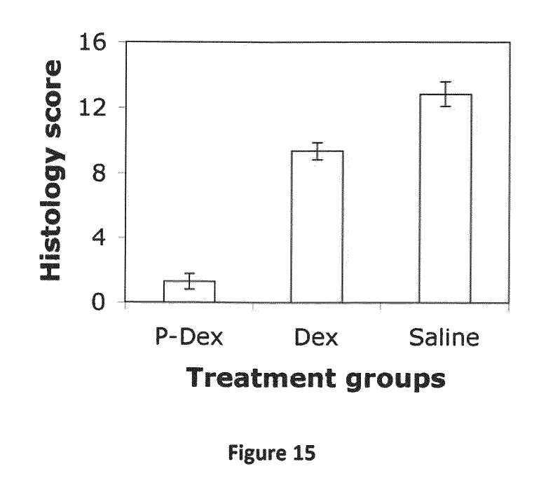

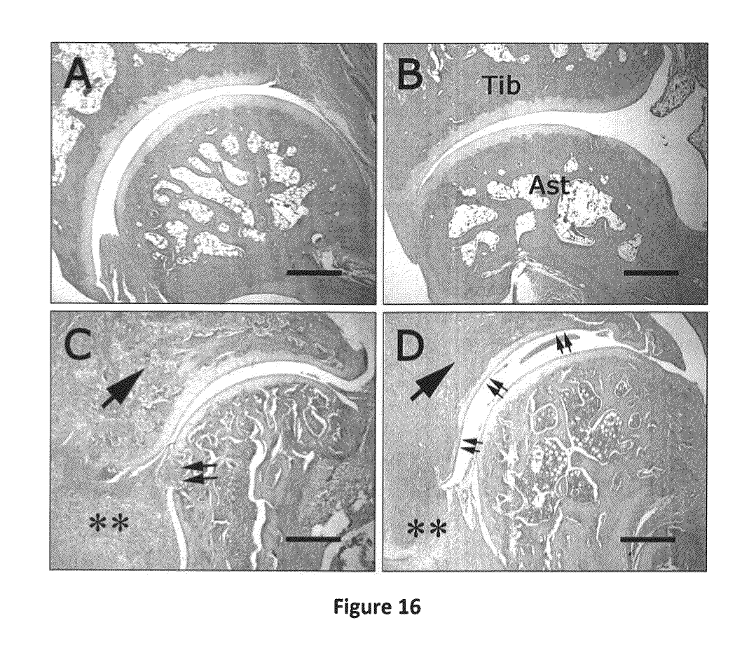

As shown in FIG. 15, the saline group was found with the highest histological score of 12.8, indicating severe inflammation and joint destruction. It was followed by free Dex group with a slightly lower score of 9.3. P-Dex treatment, on the other hand, shows the lowest histology score of 1.3, which is very close to the score of 0 of the healthy reference group. FIG. 16 composes of photomicrographs of representative H & E sections from each group. All animals from the saline treated group showed the highest scores in each parameter of histological grading. Severe bone destruction of the distal tibia and cartilage erosion of the joint are present. In the free Dex treatment group, synovial cell lining and villous hyperplasia are moderate in most of the cases. Polymorphonuclear leukocyte infiltration into periarticular soft tissue fluctuated between mild and moderate levels. Bone and cartilage destruction was present in all cases.

Compared to these two groups of animals, P-Dex treatment demonstrates the best anti-inflammatory effect with profound bone and cartilage protection.

Polymorphonuclear leukocyte infiltration into periarticular soft tissue, cellular infiltration of cartilage, cellular infiltration and bone destruction at the distal tibia are absent in most cases, which is similar to the healthy control group. Thin rim of synovial cell lining with mildly increased cellular activity was observed, suggesting the presence of minor synovitis.

### DISCUSSION

Development of drug delivery strategies for the improved treatment of rheumatoid arthritis is an emerging research area. Instead of searching for new molecular targets, it seeks to improve the therapeutic efficacy and safety profile of current available therapies. Due to the angiogenesis and leaky vasculature associated with inflammatory joints, macromolecules and colloidal vesicles can extravasate preferentially at arthritic joints (Boerman et al. (1997) Ann. Rheum. Dis. 56:369-373; Wang et al. (2004) Pharm. Res., 21:1741-1749). Based on this pathological arthrotropism, long-circulating liposomes have been proposed as a vehicle to deliver glucocorticoids to arthritic joints for the treatment of RA (Schmidt et al. (2003) Brain 126:1895-1904; Metselaar et al. (2003) Arthritis Rheum., 48:2059-66; Metselaar et al. (2004) Ann. Rheum. Dis., 63:348-353; Avnir et al. (2008) Arthritis Rheum., 58:119-129). Similarly, it has been found that synthetic water-soluble polymers such as HPMA copolymers could also passively accumulate in arthritic joints. The single administration of a pH-sensitive HPMA copolymer-Dex conjugate was able to suppress joint inflammation for 10 days (Wang et al. (2007) Arthritis Res Ther. 9:R2). Between these two delivery systems, liposome formulation is certainly easier to prepare. The pH-sensitive HPMA copolymer-Dex conjugate, on the other hand, has much better control in drug activation and release because the drug is covalently conjugated to the polymer carrier via hydrazone bond that can be activated at the arthritic joints via local acidic environment (acidosis and/or lysosome).

Polymer analogous reaction was used in the initial synthesis of HPMA copolymer-Dex conjugates (Wang et al. (2007) Arthritis Res Ther. 9:R2). While it was proven to be a very simple strategy, the inherited inconsistency of drug loading from batch to batch and residual pendent functionalities (—COOH and —CONHNH₂) of the method may impede the translation of this promising therapy into clinical applications. To resolve these problems, a new synthetic route was designed. First, a pH-sensitive Dex-containing monomer, MA-Gly-Gly-NHN=Dex was synthesized (Scheme 1). It was copolymerized with HPMA (Scheme 2). By doing this, it was possible to precisely control the drug-loading ratio and completely avoid the residual pendent functionalities.

The synthesis of MA-Gly-Gly-NHNH₂ was done by hydrazinolysis of the ethyl ester of MA-Gly-Gly-OH at room temperature with simple purification workup. However, the synthesis of MA-Gly-Gly-NHN=Dex has proven to be very difficult because both the reactant and the product can polymerize prematurely. To dissolve both Dex and MA-Gly-Gly-NHNH₂ for the reaction, DMF was first selected as the solvent with acetic acid as catalyst. After a 3-day reaction at room temperature, no product was formed even at an 1:1 molar ratio of catalyst to reactant. When the reaction temperature was elevated to accelerate the reaction, polymerization occurred. When HCl was tested as the catalyst, several byproducts were formed, which cause the purification to be extremely difficult. After several trials, methanol was selected as the best reaction solvent. MA-Gly-Gly-NHN=Dex was obtained as a single product at room temperature with acetic acid as the catalyst. Flash column chromatography was used to separate the product from the reactants. The yield was only at 30%, which needs further improvement. NMR spectra confirmed that C-3 carbonyl groups of Dex formed hydrazone with MA-Gly-Gly-NHNH₂. Due to the asymmetric structure of Dex, its conjugation to MA-Gly-Gly-NHNH₂ via hydrazone bond causes the formation of syn/anti diastereomers of MA-Gly-Gly-NHN=Dex (Scheme 3). Nevertheless, cleavage of the hydrazone bonds in the diastereomers would both produce Dex. Therefore, the MA-Gly-Gly-NHN=Dex diastereomers were not further separated but used directly in the next step to synthesize HPMA copolymer-Dex conjugate.

HPMA copolymers are biocompatible, nonimmunogenic and nontoxic. It is the most extensively studied drug carrier, with more than half of all polymeric drug conjugates in clinical evaluations based on this polymer (Duncan, R. (2003) Nat. Rev. Drug Discov., 2:347-360; Kratz et al. (2007) Expert Opin Investig Drugs 16:1037-58; Duncan, R. (2007) Biochem Soc Trans., 35:56-60). HPMA copolymers are generally prepared by free radical copolymerization and have a regular PDI of 1.5-1.6. Due to the impact this wide polydispersity may have on pharmacokinetics, safety and efficacy of polymer therapeutics, a narrower PDI is very desirable. Recently, reversible addition-fragmentation transfer (RAFT) polymerization has emerged as an alternative controlled radical polymerization technique because it can lead to the synthesis of many well-defined polymers with predictable molecular weights, and it works very well with most acrylic derivatives including acrylic acid (Chiefari et al. (1998) Macromolecules 31:5559-5562; Yanjarappa et al. (2006) Biomacromolecules 7:1665-1670). Several carboxyl-terminated trithiocarbonates have been developed as novel RAFT agents (Lai et al. (2002) Macromolecules 35:6754-6756) and used in RAFT polymerization of different monomers, including HPMA (Pan et al. (2008) Mol. Pharm., 5(4):548-58). These trithiocarbonates have extremely high chain-transfer efficiency and can yield polymers with narrow polydispersity and predictable molecular weights. In this study, RAFT polymerization was used to copolymerize MA-Gly-Gly-NHN=Dex and HPMA with S,S′-bis(α,α′-dimethyl-α″-acetic acid)-trithiocarbonate as the RAFT agent. After extensive optimization of the polymerization conditions, the M_{w} of P-Dex obtained for the animal studies was 34 kDa with a PDI of 1.34, which is significantly lower than traditional free radical copolymerization of HPMA. The incorporation efficacy of MA-Gly-Gly-NHN=Dex into P-Dex is about 50%. This conversion ratio remained constant when the feed-in ratio of the Dex monomer was changed. Thus, the drug loading in the HPMA conjugate can be precisely controlled and repeated simply by maintaining the monomer feed-in ratio. Further improvement is still needed to increase the Dex monomer conversion ratio while retaining the narrow PDI of the copolymer conjugate.

For the newly synthesized P-Dex, the same pH-sensitivity was determined as the HPMA copolymer-Dex conjugate synthesized previously with polymer analogous reaction. This feature is critical for the delivery system, as it will allow for the activation and release of Dex in the arthritic joints, where joint acidosis and the low pH of lysosomes will act as the local activation triggers. As can be seen in FIG. 12, the release of Dex from the conjugate was indeed pH-sensitive. At pH 5.0, the Dex release from the conjugate is of zero order. This is similar to the previous results from: HPMA copolymer-Dex conjugate synthesized with polymer analogous reaction. The release rate is around 1% per day. Under neutral condition, the release of Dex from P-Dex is extremely low, which ensures that the drug conjugate would not be activated prematurely in the circulation.

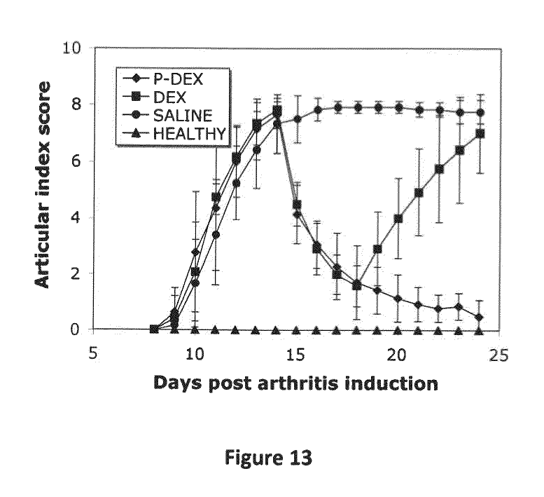

To test the therapeutic efficacy of P-Dex in vivo, P-Dex was administer to AIA rats on days 14^{th} post arthritis induction as a single bolus i.v. administration. Equivalent dose of free Dex was made into 4 aliquots and administered i.p. on days 14-17. Saline group and healthy group were used as two control groups. As can be seen in FIGS. 12 and 13, both AI score and the ankle diameter showed dramatic decreases after the initiation of the P-Dex and free Dex treatments, which can be attributed to the well-known potency and fast action of GCs. This result indirectly suggests that the activation of P-Dex is immediate, due to the local acidic environment in arthritic joints. The activation may also happen post endocytosis in the lysosomes of macrophage-like synoviocytes. The two treatment groups started to differentiate right after the last dose of free Dex. Arthritis flare happened immediately in the free Dex group, while the P-Dex group continued the healing process until the end of the study. At the end point, the AI score and ankle diameter of the free Dex group is at the same level as the saline group. The P-Dex group, on the other hand, is very similar to the healthy group. Compared to free Dex, the anti-inflammatory effect of P-Dex is clearly much longer. The retention of the polymeric drug conjugate in arthritic joints (Wang et al. (2004) Pharm. Res., 21:1741-1749) and the gradual activation (low pH mediated) of Dex may together contribute to the observed superior anti-inflammatory effect of P-Dex.

Bone and cartilage destruction are often associated with RA joints. To investigate if P-Dex would preserve bone, endpoint BMD evaluation of the arthritic joints from different treatment groups was performed. From FIG. 14, it is obvious that both free Dex and P-Dex treatments provide bone preservation when compared to the saline group. This may be attributed to their immediate suppression of joint inflammation after administration. This result clearly echoes the disease modifying effects of low dose glucocorticoids observed in human clinical trials (Kirwan, J. R. (1995) N Engl J Med 333:142-146). The mean BMD of the P-Dex treated group is higher than the group treated with free Dex group, suggesting that the prolonged suppression of inflammation by P-Dex offers better protection to the bone.

Synovial hypertrophy and villous hyperplasia is the major histopathological characteristics in the early stages of RA. The ongoing synovitis, especially the invasive pannus tissue leads to the destruction of the joint cartilage and bone if effective treatment is not initiated. To gain a better understanding of different treatments on joint inflammation at the microscopic level, the isolated joints were decalcified, sectioned and evaluated histologically (FIGS. 15 & 16). The histological grading showed dramatic difference among the treatment groups. While the free Dex group seems to offer some clinical improvement, it is not comparable to the strong and long lasting anti-inflammatory and joint protective benefits provided by the P-Dex treatment. It is unknown if the very mild synovial cell lining and villous hyperplasia observed in P-Dex group is the result of an ongoing healing process or the prelude of the arthritic flare.

From the data obtained, it is obvious that the P-Dex has a superior and long lasting therapeutic effect for the treatment of RA when compared to free Dex. It was also confirmed that the therapeutic efficacy of the newly synthesized P-Dex was comparable to the HPMA copolymer-Dex conjugate synthesized previously using polymer analogous reaction (Wang, et al. (2007) Arthritis Res Ther., 9:R2). As a unique advantage, the new P-Dex has a well-define chemical structure, which will facilitate its translation into clinical applications.

While further development of the new P-Dex is very promising, many questions still remain. The huge potential of P-Dex in reducing the systemic side effects of GCs have not been fully investigated due to the lack of a proper animal model; The mechanism of long lasting therapeutic benefits of P-Dex needs to be elucidated; The study was ended 10 days post treatment initiation due to the concern of the welfare of the saline group. Therefore, it is still not clear how long the therapeutic effect of P-Dex will last beyond this 10 days period. This question is clinically relevant because a once-a-month infusion will certainly have a better patients' compliance than a once-a-week infusion.

Thus, a novel pH-sensitive Dex-containing monomer (MA-Gly-Gly-NHN=Dex) was designed, synthesized and copolymerized with HPMA using RAFT copolymerization. The resulting P-Dex has a well-defined structure, controllable molecular weight and low PDI. The Dex loading in the conjugate can be simply controlled by adjusting monomer feed-in ratio. The in vivo evaluation showed that the newly synthesized P-Dex offers superior and longer-lasting anti-inflammatory effects when compared to free Dex. This finding confirms the previous finding with HPMA copolymer-dexamethasone conjugate synthesized via polymer analogous reactions. The development of this well-defined polymer-drug conjugate is one step further to its clinical application. Additional research efforts are warranted to elucidate its full therapeutic potential.

### Example 2

### Synthesis of poly[N-(2-hydroxypropyl)methacrylamide-co-N-(3-Aminopropyl)methacrylamide hydrochloride][poly(HPMA-co-APMA)]

HPMA (1 g, 6.98 mmol), APMA (12.5 mg, 0.07 mmol), 2,2′-azobisisobutyronitrile (AIBN, 0.057 g) and 1 μL 3-mercaptopropionic acid (3-MPA) were dissolved in 8 mL methanol in an ampule. After 5 minutes bubbling the ampule was sealed. The mixture was then kept at 50° C. for 24 hours. Then the mixture was precipitated in 150 mL acetone for three times and vacuum dried at 30° C. The resulting poly(HPMA-co-APMA) was further purified by LH-20 column chromatography. The amine content of the copolymer was determined by ninhydrin assay as 5.5×10⁻⁵ mol/g.

Synthesis of poly(HPMA-co-APMA)-IRDye 800 CW conjugate

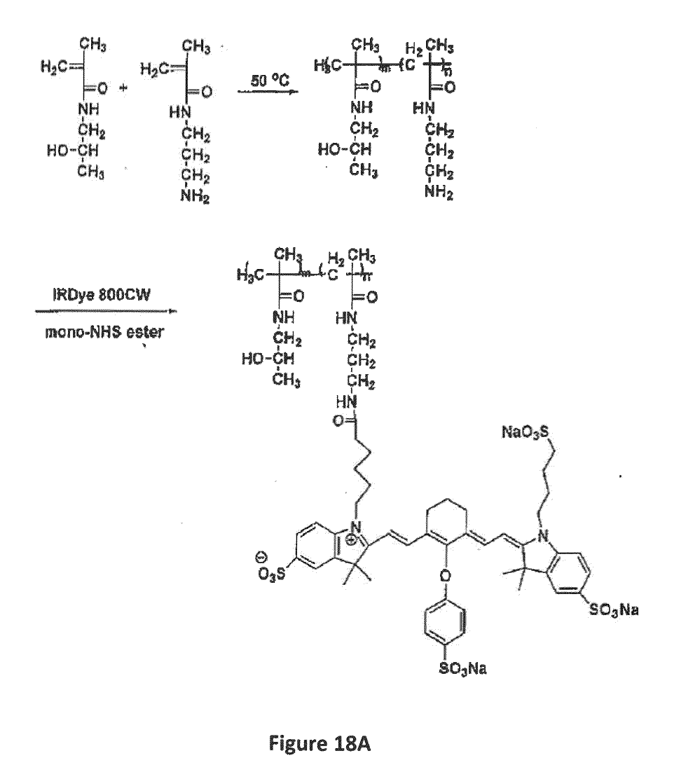

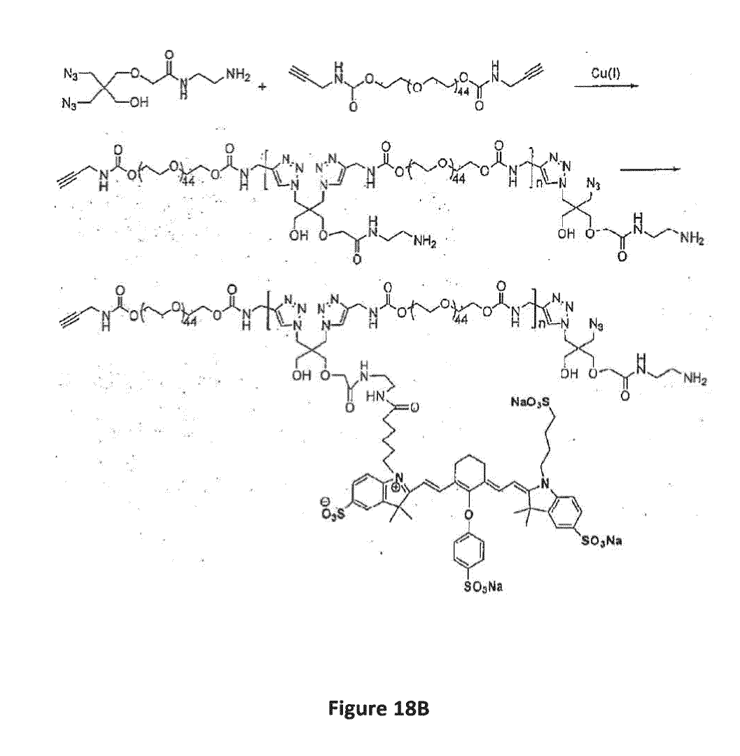

Poly(HPMA-co-APMA) (31 mg, 0.0017 mmol), IRDye 800 CW (1 mg, 0.00086 mol, LI-COR® Biosciences, Lincoln, Nebr.) was dissolved in about 600 μL of dimethylformamide (DMF) and 15 μL N,N-diisopropylethylamine (DIPEA) was added. The mixture was stirred overnight in darkness at room temperature. After that, the mixture was directly purified on LH-20 column. The polymer fraction was collected. The solvent was removed by evaporation, then the sample was dissolved in ddH₂O, dialysis and freeze-drying. See FIG. 18A.

### Optical Imaging of Inflammatory Arthritic Joints

Similar to the HPMA copolymer approach, optical polymer contrast agents and therapeutic agent comprising polymer can also be synthesized via the click PEG chemistry (FIG. 18B).

### Optical Imaging of Inflammatory Arthritic Joints

1) Dissolve 1 mg polymer-IRdye conjugate into 200 μL saline, then filtered through 0.2 μm Nalgene™ syringe filter.

2) IVIS imaging was performed using ICG filter (Excitation wavelength 784 nm, emission wavelength 830 nm), which most matches the IRdye (Excitation wavelength 774 nm, emission wavelength 789 nm).

3) Pictures were taken before the polymer conjugate was injected to offset the autofluorescence.

4) Serum transfer arthritis mouse was injected with the polymer-IRdye conjugate via tail vein.

5) NIR pictures were taken at 15 minutes, 1 hour, 3 hours, 6 hours, 9 hours, and 24 hours post injection.

This example describes synthesized macromolecular near infrared optical imaging contrast agents as a cheap and convenient tool for clinical diagnosis and treatment evaluation of tumors, inflammatory diseases and other diseases associated with enhanced vascular permeability. Examples of the diseases can benefit from this tool include, without limitation, rheumatoid arthritis, systemic lupus erythematosus, breast cancer, skin cancer, Crohn's disease, and colon cancer. For imaging of the disease lesions associated with or close to the body exterior surface, a simple black box equipped with near infrared light source and CCD camera would be sufficient. For imaging of lesions associated with GI tract, colon, vagina, and other internal organs or tissues, endoscope-assisted optical imaging can be used. Compared to current diagnosis methods such as MRI or CT, the instant technology is simpler and costs much less. While at present, the near infrared dye molecules are conjugated to macromolecular carrier via non-degradable bond, peptide sequences can be used as the linker. By using peptide sequences that are specific to different enzymes, the macromolecular optical imaging contrast agent will allow reporting the local biochemical environment at the lesion.

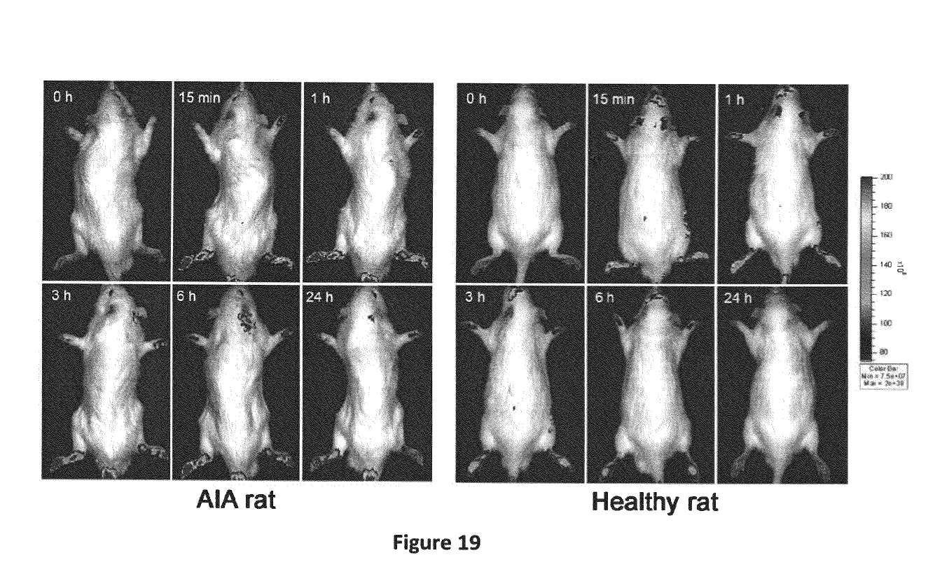

The instant study was performed on a rheumatoid arthritis animal model. As shown in FIG. 19, adjuvant-induced arthritis (AIA) rat and healthy rat showed distinctive optical imaging pattern after administration of the macromolecular optical imaging contrast agent. It demonstrates the use of the contrast agents and the novel concept of using them in diagnosis and treatment evaluation of different diseases.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method of treating an inflammatory disease in a subject in need thereof comprising administering to the patient a composition comprising at least one N-(2-hydroxypropyl)methacrylamide (HPMA) copolymer and at least one pharmaceutically acceptable carrier,
wherein said HPMA copolymer comprises an anti-inflammatory therapeutic agent conjugated to the copolymer via a pH-sensitive linker.

2. The method of claim 1, wherein said an anti-inflammatory therapeutic agent is dexamethasone.

3. The method of claim 1, wherein said pH-sensitive linker comprises a hydrazone.

4. The method of claim 1, wherein said HPMA copolymer has the structure: 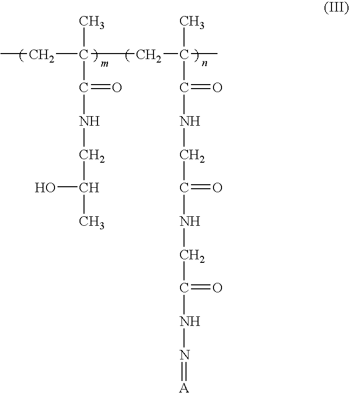
wherein A is a therapeutic agent; and wherein m and n are independently from about 1 to about 1000.

5. The method of claim 4, wherein said HPMA copolymer is

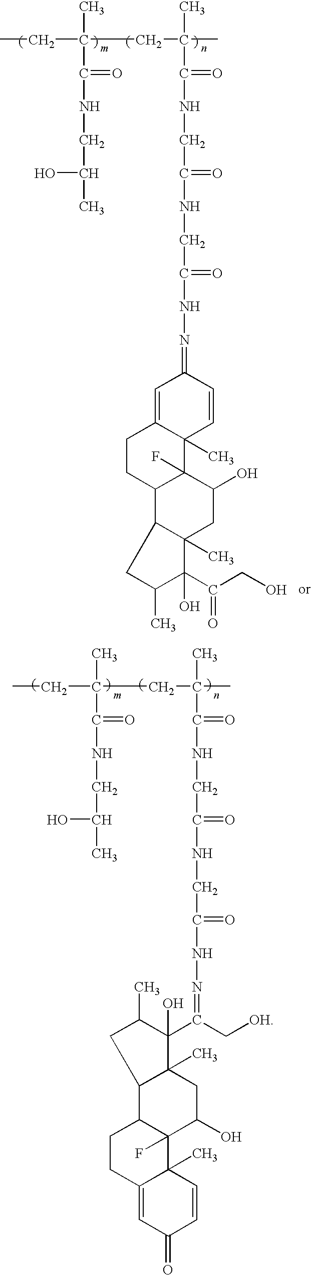

6. The method of claim 1 further comprising the administration of at least one additional anti-inflammatory therapeutic agent.

7. A compound having the structure: 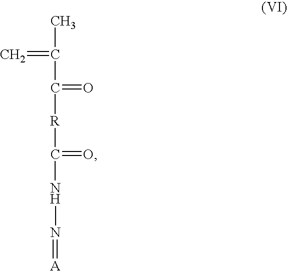
wherein A is a therapeutic agent and R is a linker.

8. The compound of claim 7, wherein R is selected from the group consisting of an alkyl, an aryl, and a peptide.

9. The compound of claim 7, wherein said therapeutic agent is dexamethasone.

10. A method of synthesizing a copolymer, said method comprising polymerizing the compound of claim 7 with a second monomer.

11. The method of claim 10, wherein said second monomer has the structure: 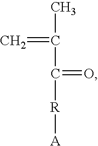
wherein R is a linker and A is an imaging agent or a therapeutic agent, and wherein A is optional.

12. The method of claim 10, wherein said polymerization is reversible addition fragmentation transfer (RAFT) polymerization.

13. The method of claim 10, wherein said HPMA copolymer has the structure: 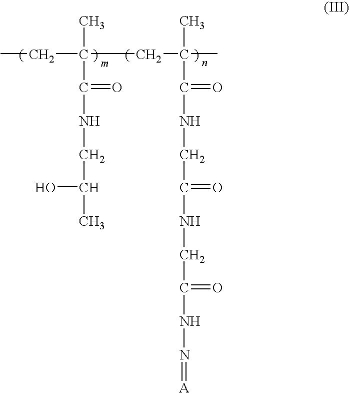
wherein A is therapeutic agent; and wherein m and n are independently from about 1 to about 1000.

14. The method of claim 10, wherein said HPMA copolymer is

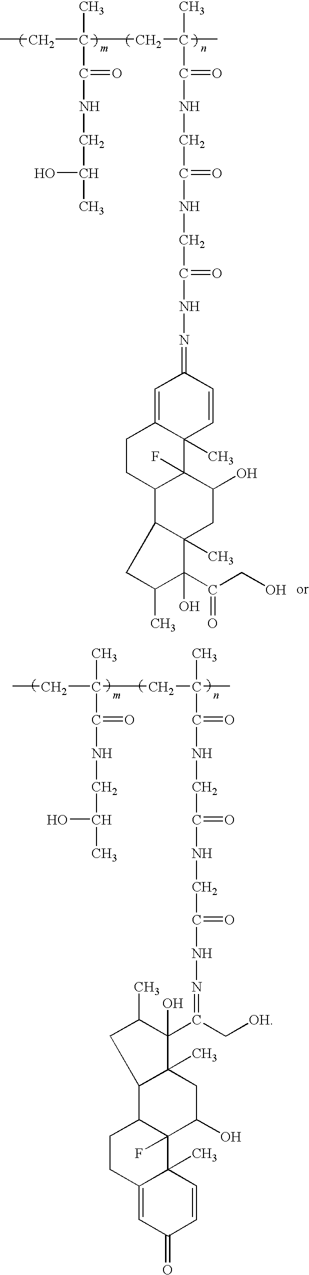

15. A method of imaging an inflammatory disease in a subject, said method comprising administering to the patient a composition comprising at least one N-(2-hydroxypropyl)methacrylamide (HPMA) copolymer and at least one pharmaceutically acceptable carrier,
wherein said HPMA copolymer comprises at least one imaging agent conjugated to the copolymer via a linker.
